(19) United States
(12) Patent Application Publication
(10) Pub. No.: US 2010/0010082 A1
(43) Pub. Date: Jan. 14, 2010

(54) FORMULATIONS FOR TREATING EYE DISORDERS

(75) Inventors: Eddie CHONG, North Vancouver (CA); Clive BURGE, Brentwood Bay (CA); Lee MIZZEN, Victoria (CA)

Correspondence Address: DECHERT LLP, P.O. BOX 390460, MOUNTAIN VIEW, CA, 94039-0460, US

(73) Assignee: Aspreva International Ltd., Victoria (CA)

(21) Appl. No.: 12/500,399

(22) Filed: Jul. 9, 2009

Related U.S. Application Data
(60) Provisional application No. 61/079,413, filed on Jul. 9, 2008.

(51) Int. Cl. A61K 31/365; C07D 307/87; A61P 27/02

(52) U.S. Cl. 514/470; 549/310

(57) ABSTRACT

The present disclosure relates to ophthalmic solutions and methods of using the solutions to treat ocular disorders

### 1. CROSS-REFERENCES TO RELATED APPLICATIONS

This application claims benefit under 35 U.S.C. § 119(e) of application Ser. No. 61/079,413, filed Jul. 9, 2008, the contents of which are incorporated herein by reference.

### 2. BACKGROUND

Many inflammatory diseases of the eye occur de-novo or as secondary complications to various systemic diseases such as autoimmune diseases or infections. Standard treatments, such as the use of topically applied steroids, are directed to controlling the inflammatory symptoms in the eye. However, a complication with steroid treatments is that a significant percentage of treated subjects suffer from increased intraocular pressure, which can exacerbate eye disorders, such as glaucoma and cataracts. In some instances, the ocular disorder is refractory to the effects of the topically applied steroid.

In some instances, systemic treatments with anti-inflammatory steroids or other immunosuppressive agents are used to treat the ocular inflammation. However, adverse effects from systemic treatments can limit their use. Side effects can include hypertension, hyperglycemia, peptic ulceration, osteoporosis, growth limitation, myopathy, and kidney dysfunction. Even systemic steroid therapies also have potentially sight-threatening side effects such as glaucoma, cataract and susceptibility to eye infection. Some alternative therapies, for example, topical administration of cyclosporine A (Restasis™, Allergan Inc.) (Tauber. J., 1998, Adv Exp Med Biol. 438:969-72) have been approved for use in the treatment of certain ocular disorders. However, topically applied cyclosporine A (CsA) is indicated as being poorly tolerated and having low bioavailability (Lallemand et al., 2003, Eur J Pharm Biopharm. 56(3):307-18). Thus, it is desirable to find other therapies that can be used to treat ocular disorders associated with inflammatory conditions and autoimmune diseases.

Citations of the above documents, and in this application, is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are hereby incorporated in their entirety by reference.

### 3. SUMMARY

The present disclosure relates to ocular solutions for treating various eye disorders associated with inflammatory and autoimmune conditions. In one aspect, the ocular solution has a composition consisting essentially of sodium mycophenolic acid (NaMPA), where the pH of the solution is from about pH 6.0 to 8.5. Although the NaMPA is highly soluble in aqueous solutions, the MPA in the ocular solution is found to penetrate into the eye to achieve levels sufficient to have therapeutic benefit. In some embodiments, the ocular solutions has a composition consisting essentially of NaMPA and one or more additives selected from a preservative, viscosity enhancing agent, wetting agent, buffering agent, lubricating agent, antioxidant, and tonicity agent. The levels of NaMPA can be up to the solubility limits of the drug in aqueous solution at the indicated pH ranges. In some embodiments, the amount of NaMPA in the solution can be up to 4.5% w/v. In various embodiments, the levels of sodium in the ocular solution can be from 0.4 to 2.0% w/v. In some embodiments, sodium levels above the isotonic condition (e.g., equivalent to 0.9% NaCl) can be used.

In some embodiments, the ocular solution includes NaMPA and a preservative. In particular, the preservative is EDTA, which can be present from about 0.005 to about 0.050% w/v, 0.005 to about 0.040% w/v, 0.010 to about 0.030% w/v, 0.010 to about 0.020% w/v, or from about 0.010 to about 0.015% w/v. In some embodiments, the EDTA can be present at 0.005, 0.01, 0.012, 0.014, 0.016, 0.018, 0.020, 0.030, 0.040, or 0.050% w/v. In some embodiments, the EDTA (as disodium dehydrate) is present at about 0.012% w/v.

In some embodiments, the ocular solution includes NaMPA, a preservative and a buffering agent. An exemplary formulation of this type can include the preservative EDTA, in amounts as noted above; a buffering agent of borate or tromethamine, with an amount of buffer to provide a buffering capacity of 0.01 to about 0.1; and a solution pH of about 7.0 to 8.0.

The ocular solution can be used to treat various de-novo inflammatory eye disorders or those associated with autoimmune diseases or infections affecting the eye. In some embodiments, these eye conditions include “front of the eye” disorders such as blepharitis; keratitis; rubeosis iritis; Fuchs' heterochromic iridocyclitis; chronic uveitis or anterior uveitis; conjunctivitis; allergic conjunctivitis (including seasonal or perennial, vernal, atopic, and giant papillary); keratoconjunctivitis sicca (dry eye syndrome); iridocyclitis; iritis; scleritis; episcleritis; corneal edema; scleral disease; ocular cicatrcial pemphigoid; pars planitis; Posner Schlossman syndrome; Behçet's disease; Vogt-Koyanagi-Harada syndrome; hypersensitivity reactions; conjunctival edema; conjunctival venous congestion; periorbital cellulitis; acute dacryocystitis; non-specific vasculitis; and sarcoidosis. In some embodiments, the eye conditions include “back of the eye” disorders such as macular edema; angiographic cystoid macular edema; retinal ischemia and choroidal neovascularization; macular degeneration; retinal diseases (e.g., diabetic retinopathy, diabetic retinal edema, retinal detachment); inflammatory diseases such as uveitis (including panuveitis) or choroiditis (including multifocal choroiditis) of unknown cause (idiopathic) or associated with a systemic (e.g., autoimmune) disease; episcleritis or scleritis; Birdshot retinochoroidopathy; vascular diseases (e.g., retinal ischemia, retinal vasculitis, choroidal vascular insufficiency, choroidal thrombosis); neovascularization of the optic nerve; and optic neuritis.

The ocular solutions can be applied at doses sufficient to provide a therapeutic benefit. In some embodiments, the ocular solution can be applied topically to the affected eye one to eight times per day. In some embodiments, the ocular solutions can be administered once or two times per day. In some embodiments, the ocular solutions can be applied once every two days, once every four days, or once a week as needed to treat the ocular disorder.

### 4. BRIEF DESCRIPTION OF THE FIGURES

FIG. 1 shows studies on ocular tissue penetration of NaMPA and cyclosporine achieved in rabbits following topical administration to the eye 8 times daily for 14 days. In these studies, animals received either NaMPA or cyclosporine as topical solutions applied to both eyes. Tissues were harvested for analysis at the end of drug dosing, on day 14. Data are expressed as ηg drug per gram (ηg/g) of ocular tissue. These studies demonstrated that the NaMPA formulation penetrated all ocular tissues examined, including anterior tissues (e.g., conjunctiva, lacrimal sac, aqueous humor) and posterior tissues (e.g., retina/choroid), in particular, the aqueous humor; iris/ciliary body; lachrymal sac; sclera; and retina/choroid.

FIG. 2 shows the levels of NaMPA or cyclosporine measured in ocular tissues, expressed as a ratio, derived from the results described in FIG. 1.

FIGS. 3A, 3B and 3C show the ocular tissue penetration data for the MPA salts (1%, 2%, 4% w/v) for the combined 1-day studies: NaMPA; NaMPA+borate; tromethamine MPA; and morpholine MPA. Note that concentrations are given in micrograms/mL. One animal per treatment group was randomly selected, euthanized, and both eyes harvested for determining MPA levels (average of both eyes taken). The cyclosporine data is not presented in this table.

FIG. 4 shows the tear break up time (TBUT) values in rabbits where dry eye was induced by bilateral lacrimal gland injection of concanavalin A (Con A). Results are shown for rabbits treated with NaMPA or Vehicle from Day 0 to Day 17. Con A was injected on Day 8. Induction of dry eye was observed as measured by a reduction in tear break up time (TBUT) values over Days 9-12. Statistically significant increases in TBUT values (e.g., Days 14-17) are indicated with an asterisk for the NaMPA groups vs. Vehicle.

FIG. 5 shows the TBUT values for the Restasis®, dexamethasone and Vehicle groups from the same study as described in FIG. 4. Statistically significant increases in TBUT values (e.g., Days 14-17) are indicated with an asterisk for Restasis® or dexamethasone groups vs. Vehicle.

FIG. 6 shows the clinical scoring for conjunctival hyperemia, chemosis, discharge and lid edema graded on a 0-4 scale (see Grading Systems for Allergic Response) in animals systemically sensitized on Day 0 to short ragweed allergen (SRW) then given a topical ocular challenge on Day 27 with SRW. Scoring was done 15 minutes after SRW challenge. Groups were treated on Days 21 to 27 with NaMPA, Pred Forte® (prednisolone acetate) or Vehicle or left untreated. Statistically significant reductions in conjunctival hyperemia were seen for the 2%, 1% and 0.5% NaMPA groups and the Pred Forte® group vs. negative control groups. There was also a statistically significant reduction in chemosis for the Pred Forte® group vs. negative control groups.

FIG. 7 shows the clinical scoring for itching/face washing behaviour 3, 5, 7 and 10 minutes after SRW challenge for the same groups of animals shown in FIG. 6. Statistically significant reductions were seen at the 10 minute interval for the 2% and 1% NaMPA groups vs. negative controls.

FIG. 8 shows the number of infiltrating CD4+ cells (CD4+ T cells) viewed by light microscopy in conjunctival tissue for the same animals as depicted in FIGS. 6 and 7, that were sacrificed on Day 27 after clinical scoring was done. Immunostaining for CD4+ cells was done by standard procedures as described in Studies Based on Ragweed Induced Allergic Conjunctivitis. Statistically significant reductions were seen for the 2% NaMPA and Pred Forte® groups vs. negative controls.

FIG. 9 shows the number of infiltrating macrophages viewed by light microscopy in conjunctival tissue for the same tissue samples as described in FIG. 8. Immunostaining for macrophages was done as described in Studies Based on Ragweed Induced Allergic Conjunctivitis. Statistically significant reductions were seen for the 2% NaMPA and Pred Forte® groups vs. negative controls.

FIG. 10 shows the clinical scoring for conjunctival hyperemia in animals 5, 10, 15, 20 and 30 minutes after topical ocular challenge with compound 48/80. Animals were treated with NaMPA, Pred Forte® or Vehicle or left untreated from Days 1-7, then challenged on Day 7 with compound 48/80. Statistically significant reductions were seen for the 1% NaMPA group vs. the untreated group at the 15 and 20 minute intervals.

FIG. 11 shows the clinical scoring for discharge in animals 5, 10, 15, 20 and 30 minutes after challenge with compound 48/80 for the same groups of animals depicted in FIG. 10. Statistically significant reductions were seen in the 2% and 1% NaMPA groups and the Pred Forte® group vs. controls at the 20 or 30 minute time intervals.

FIG. 12 shows the clinical scoring for chemosis 5, 10, 15, 20 and 30 minutes after challenge with compound 48/80 for the same groups of animals depicted in FIGS. 10 and 11. Statistically significant reductions were seen in the 2% NaMPA and Pred Forte® groups vs. Vehicle control at the and/or 30 minute time interval.

### 5. DETAILED DESCRIPTION

Delivery of drugs to the eye are challenging given the anatomical and physiologic barriers that limit ocular access of drug compounds into the eye, such as low corneal permeability. In order to enhance bioavailability, it has been suggested that the drug be lipid soluble to enhance penetration through the cornea and the lipophilic endothelium (Ahmed et al., 1987, “Physicochemical determinants of drug diffusion across the conjunctiva, sclera, and cornea,” J Pharm Sci. 76: 583-586; Wang et al., 1991, “Lipophilicity influence on conjunctival drug penetration in the pigmented rabbit: a comparison with corneal penetration,” Curr Eye Res 10: 571-579). For lipophilic molecules that have poor solubility in aqueous solutions, e.g., steroids, complexes to drug carriers such as cyclodextrins have been used to solubilize and deliver the drug to the membrane surface where they can partition into the lipophilic membrane from the carrier molecule (Loftsson T and Masson M, 2001, “Cyclodextrins in topical drug formulations: theory and practice,” Int J Pharm 212: 29-40).

The immunosuppressive compounds mycophenolic acid (MPA) and its ester prodrug form, mycophenolate mofetil (MMF) have been mainly used to prevent rejection of allogenic organ transplants and for treatment of certain autoimmune diseases, such a systemic lupus erythematosus and myasthenia gravis. MPA is known to specifically inhibit the enzyme inosine monophosphate dehydrogenase (IMPDH), which is used preferentially by T and B cells to generate de novo guanosine nucleotides required for cell replication. Approved prescription drug versions of MMF (CellCept®) and an enteric-coated sodium salt of MPA (Myfortic®) are marketed for prevention of solid organ transplant rejection. Both are given orally to achieve systemic immunosuppression. In addition, MMF and MPA are known to possess other biological effects, including those that are anti-inflammatory. Because of its immunosuppressive and anti-inflammatory effects, orally administered MMF has been tested as a treatment for certain eye disorders, such as uveitis and refractory inflammatory eye disease (Zierhut et al., 2005, “MMF and eye disease,” Lupus 14 Suppl 1:s50-4; Choudhary et al., 2006, J Ocul Pharmacol Ther. 22(3):168-75). Formulations of MMF for topical administration to the eye have been recently described (Knapp et al., 2003, J Ocul Pharmacol Ther. 19(2): 181-92). Ocular solutions containing at least one macrolide and/or mycophenolic acid is described in the PCT application publication WO2005/030305A1. MMF is more lipophilic than MPA, being soluble in alcohol and only slightly soluble in water (CellCept® label), while the sodium salt of MPA is indicated as being highly soluble in aqueous solutions at physiological pH (Myfortic® label). To increase the bioavailability of MMF in the eye, it has been formulated with cyclodextrins (Knapp, supra).

It has now been found by the inventors that the sodium salt of MPA formulated at physiological pH is effective in penetrating anterior and posterior eye structures when applied topically to the eye. The MPA levels achieved within the eye structures with this formulation can be at levels sufficient to have a therapeutic benefit. The penetration into the eye occurs even though the sodium salt of MPA is highly soluble in aqueous solutions at physiological pH and is significantly less lipophilic than MMF. Accordingly, the disclosure provides ocular solutions containing mycophenolic acid and methods of using the formulations to treat various ocular disorders. Preferably, the disclosure provides formulations containing the sodium salt of MPA to treat various ocular disorders.

For the descriptions provided in this specification and the appended claims, the singular forms “a”, “an” and “the” include plural referents unless the context clearly indicates otherwise. Thus, for example, reference to “an agent” includes more than one agent, and reference to “a compound” refers to more than one compound.

It is to be further understood that where descriptions of various embodiments use the term “consisting essentially of,” those skilled in the art would understand that in some specific instances, an embodiment can be alternatively described using language “consisting of.”

It is to be understood that both the foregoing general description, including the drawings, and the following detailed description are exemplary and explanatory only and are not restrictive of this disclosure.

In some embodiments, the ocular solution is a composition consisting essentially of sodium mycophenolic acid (NaMPA), where the pH of the solution can be from about 6.0 to about 8.5. In some embodiments, the ocular solution is a composition consisting essentially of sodium mycophenolic acid, and one or more additives selected from a preservative, viscosity enhancing agent, wetting agent, buffering agent, lubricating agent, antioxidant, and tonicity agent, where the pH of the solution can be from about 6.0 to about 8.5.

The amount of NaMPA in the ocular solution can be up to the solubility limits of the drug in aqueous solution at the indicated pH range. In some embodiments, the amount of NaMPA in the ocular solution can be up to 4.5% w/v. In some embodiments, the ocular solution can have an NaMPA level of from about 0.01% w/v to about 4.5% w/v of NaMPA. In some embodiments, the ocular solution can have an NaMPA level of from about 0.1% w/v to about 4.5% w/v of NaMPA. In some embodiments, the ocular solution can have an NaMPA level of from about 0.5% w/v to about 4.5% w/v of NaMPA. In some embodiments, the ocular solution can have an NaMPA level of from about 0.01% w/v to about 4.0% w/v of NaMPA. In some embodiments, the ocular solution can have an NaMPA level of from about 0.1% w/v to about 4.0% w/v of NaMPA. In some embodiments, the ocular solution can have an NaMPA level of from about 0.5% w/v to about 4.0% w/v of NaMPA. In some embodiments, the ocular solution can have an NaMPA level of from about 0.05% w/v to about 3.0% w/v of NaMPA. In some embodiments, the ocular solution can have an NaMPA level of from about 0.1% w/v to about 3.0% w/v of NaMPA. In some embodiments, the ocular solution can have an NaMPA level of from about 0.5% w/v to about 3.0% w/v of NaMPA. In some embodiments, the ocular solution can have an NaMPA level of from about 0.1% w/v to about 2.0% w/v of NaMPA. In some embodiments, the ocular solution can have an NaMPA level of from about 0.2% w/v to about 1.0% w/v of NaMPA. In some embodiments, the ocular solution can have an NaMPA level of from about 2% to about 4% w/v of NaMPA. In some embodiments, the ocular solution of NaMPA has levels of the drug selected from 0.05, 0.06, 0.08, 0.1, 0.2, 0.5, 1.0, 1.5, 2.0, 2.5, 3.0, 3.5, or 4.0% w/v. In some embodiments, the NaMPA levels are selected from 2.0, 2.5, 3.0, 3.5, or 4.0% w/v. The levels of NaMPA selected can be based on the amounts required to achieve therapeutically beneficial levels in the eye. The sodium salt of MPA is described in, among others, WO97/38689.

In some embodiments, the pH of the ocular solution can be within 1.0 to 1.5 pH units from physiological pH, particularly the physiological pH in the external environment of the eye. The pH of human tears is approximately pH 7.4. Hence, the pH of the ocular solution can be about 1.0 to 1.5 pH units above or below pH 7.4. In some embodiments, the pH of the ocular solution is from about pH 6.0 to about pH 8.5. In some embodiments, the pH of the ocular solution is from about pH 6.0 to about pH 8.0. In some embodiments, the pH of the ocular solution is from about 6.5 to about 8.0. In some embodiments, the pH of the ocular solution is from about 7.0 to about 8.0. In some embodiments, the pH of the ocular solution is from about 7.0 to about 7.5. A person of skill in the art can select a pH that balances the stability and efficacy of the NaMPA formulation at the indicated pH and the tolerability of the eye to differences in pH from the natural condition.

In some embodiments of the ocular solutions, the total sodium level in the solution is from about 0.4 to about 2.0% w/v. In some embodiments, the total sodium in the solution is from about 0.4 to about 1.0% w/v. In some embodiments, the total sodium in the solution is from about 0.6 to about 0.9% w/v. In some embodiments, the total level of sodium in the ocular solution is selected from 0.4, 0.5, 0.6, 0.7, 0.8, 0.9, 1.0, 1.2, 1.4, 1.6, 1.8, and 2.0% w/v. In general, the level of sodium is that contributed by the NaMPA and any additional Na+ ions added to the solution from other sources, such as EDTA used as a preservative and/or NaOH used to adjust the pH of the ocular solution. In some embodiments, NaCl can be added to adjust the sodium levels. In some embodiments, the total sodium in the solution can be the amount isotonic to the natural environment of the eye. In general, the isotonicity of the lacrimal fluid corresponds to that of a 0.9% sodium chloride solution. However, the eye can tolerate values as low as that of a 0.6% sodium chloride solution and as high as that of a 2.0% sodium chloride solution without marked discomfort. The total osmolarity of tears in normal eyes have been reported between 311-350 mOsM/L (Ophthalmic Drug Delivery Systems, Ed. A Mitra, Dekker, 1993) and between 284-311 mOsM/L (Farris R., 1986; Tr. Am. Ophth. Soc. Vol LXXXIV). In some embodiments, the osmolarity can be from about 250 to about 450 mOsM/L, or about 250 to about 350 mOsM/L. In some embodiments, the higher levels of sodium, e.g., above 0.9% w/v, such as 1.0, 1.2, 1.4, 1.6, 1.8, or 2.0% w/v, can be used to increase the levels of un-ionized MPA (e.g., NaMPA) in the ocular solution.

In some embodiments, the counter ion to the sodium in solution is chloride. In some embodiments, the chloride in the ocular formulation can be from HCl, which is used to adjust the pH of the ocular solution, or from sodium chloride, which can be used to adjust the tonicity of the formulation. Other examples of chloride sources include potassium chloride. In some embodiments, various buffers, as further described below, can also be a source of other types of counterions.

In some embodiments, the ocular solutions can consist essentially of NaMPA and one or more additives such as preservatives, viscosity increasing agents, wetting agents, buffering agents, lubricating agents, antioxidants, and tonicity agents. It is to be understood that the categories of agents are not meant to be mutually exclusive such that some agents can fall into multiple categories. For example, a wetting agent can also have viscosity enhancing properties, and therefore can be a wetting agent as well as a viscosity enhancing agent.

In some embodiments, the additive can be one or more buffering agents for adjusting and/or maintaining the pH of the ocular solution at a specified pH range. Buffering agents are usually composed of a weak acid or base and its conjugate salt, where the “buffer capacity” 8 is defined as:

$$β = \frac{Δ B}{Δ pH}$$

where ΔB is the gram equivalent of strong acid/base to change pH of 1 liter of buffer solution, and ΔpH is the pH change caused by the addition of strong acid/base. The relationship between buffer capacity and buffer concentrations can be defined by the following formula:

$$β = 2.3 C \frac{Ka [ {H}_{3} {O}^{+} ]}{{( Ka + [ {H}_{3} {O}^{+} ] )}^{2}}$$

where C is the total buffer concentration (i.e., the sum of the molar concentrations of acid and salt). Generally, buffer capacity should be large enough to maintain the product pH for a reasonably long shelf-life but also low enough to allow rapid readjustment of the product to physiologic pH upon administration. Generally, buffer capacities of from about 0.01 to 0.1 can be used for ophthalmic solutions, particularly at concentrations that provide sufficient buffering capacity and minimizes adverse effects, e.g., irritation, to the eye. Exemplary buffering agents include, by way of example and not limitation, various salts (e.g., sodium, potassium, etc.), acids or bases, where appropriate, of the following: acetate, borate, phosphate, bicarbonate, carbonate, citrate, tetraborate, biphosphate, tromethamine, hydroxyethyl morpholine, and THAM (trishydroxymethylamino-methane). In some embodiments, the buffer can be present from about 0.5 mM to about 100 mM, from about 1 mM to about 50 mM, from about 1 mM to about 40 mM, from about 1 mM to about 30 mM, from about 1 mM to about 20 mM, or from about 1 mM to about 10 mM.

In some embodiments, the ocular solution of NaMPA can have one or more preservatives, for example, to extend shelf life or limit bacterial growth in the solutions during storage as well as when administered therapeutically onto the eye. Preservatives that can be used, include, among others, benzalkonium chloride, benzethonium chloride, benzododecinium bromide, cetylpyridinium chloride, chlorobutanol, ethylenediamine tetracetic acid (EDTA), thimerosol, phenylmercuric nitrate, phenylmercuric acetate, methyl/propylparabens, phenylethyl alcohol, sodium benzoate, sodium propionate, sorbic acid, and sodium perborate. The amount of preservative in the solution can be a level that enhances the shelf life, limits bacterial growth, or otherwise preserves the ocular solution, with minimal toxicity to the eye tissues (see, e.g., The United States Pharmacopeia, 22nd rev., and The National Formulary, 17th ed. Rockville, Md.: The United States Pharmacopeial Convention; pages 1692-3 (1989)). Levels of preservative suitable for use in the ocular formulations can be determined by the person skilled in the art. In some embodiments, the preservatives can be used at an amount of from about 0.001 to about 1.0% w/v. For example, the preservative can be a divalent metal ion chelator, such as EDTA, and can be from about 0.005 to about 0.050% w/v, 0.005 to about 0.040% w/v, 0.010 to about 0.030% w/v, 0.010 to about 0.020% w/v, or from about 0.010 to about 0.015% w/v. In some embodiments, the amount of preservative in the ocular solution, such as EDTA, can be about 0.005, 0.01, 0.012, 0.014, 0.016, 0.018, 0.020, 0.030, 0.040, or 0.050% w/v.

In some embodiments, the ocular solution of NaMPA can include one or more viscosity enhancing agents. The viscosity enhancing agent typically enhances the viscosity of the ocular solution to increase retention time of the solution on the eye, and in some instances, to provide a protective layer on the eye surface. Viscosity enhancing agents include, among others, carbopol gels, dextran 40 (molecular weight of 40,000 Daltons), dextran 70 (molecular weight of 70,000 Daltons), gelatin, glycerin, carboxymethycellulose (CMC), hydroxyethyl cellulose, hydroxypropyl methylcellulose, (HPMC) methylcellulose, ethylcellulose, polyethylene glycol, poloxamer 407, polysorbate 80, propylene glycol, polyvinyl alcohol, and polyvinylpyrrolodine (povidone), in various molecular weights and in various compatible combinations. Viscosity of a solution is given in poise units, with a viscosity between about 25 and 50 cps being suitable for ophthalmic solutions. The amount of agent for use in the ocular formulations can be determined by one of skill in the art, and can provide residence times in the eye of 15 min or more, 30 min or more, 1 hr or more, 2 hrs or more, 3 hrs or more, 4 hrs or more, 6 hrs or more, 8 hrs or more, 12 hr or more as would be suitable for the condition being treated and the desired retention time of the solution on the eye.

In some embodiments, the ocular solution of NaMPA can include one or more antioxidants. Suitable antioxidants, include, by way of example and not limitation, EDTA (e.g., disodium EDTA), sodium bisulphite, sodium metabisulphite, sodium thiosulfate, thiourea, and alphatocopherol.

In some embodiments, the additive is one or more wetting agents. Generally, wetting agents can hydrate and limit drying of the eye. Wetting agents generally are hydrophilic polymers, including, by way of example and not limitation, polysorbate 20 and 80, poloxamer 282, and tyloxapol. In some embodiments, wetting agents also include, among others, cellulose based polymers, such as HPMC and CMC; polyvinylpyrrolodine; and polyvinyl alcohol.

In some embodiments, the additive is one or more lubricating agents. Ocular lubricants can approximate the consistency of endogenous tears and aid in natural tear build-up. Lubricating agents can include non-phospholipid and phosphipid-based agents. Ocular lubricants that are non-phospholipid based include, but are not limited to, propylene glycol; ethylene glycol; polyethylene glycol; hydroxypropylmethylcellulose; carboxymethylcellulose; hydroxypropylcellulose; dextrans, such as, dextran 70; water soluble proteins, such as gelatin; vinyl polymers, such as polyvinyl alcohol, polyvinylpyrrolidone, povidone; petrolatum; mineral oil; and carbomers, such as, carbomer 934P, carbomer 941, carbomer 940, and carbomer 974P. Non-phospholipid lubricants can also include compatible mixtures of any of the foregoing agents.

In some embodiments, the ocular lubricating agent is a phospholipid-based lubricant. As used herein, “phospholipid lubricant” refers to aqueous compositions which comprise one or more phospholipids. Tear film has been shown to comprise a lipid layer, which is secreted by tear glands and is composed of various types of phospholipids (see, e.g., McCulley and Shine, 2003, The Ocular Surface 1: 97-106). Examples of phospholipid lubricant formulations include those disclosed in U.S. Pat. Nos. 4,804,539; 4,883,658; 4,914,088; 5,075,104; 5,278,151; 5,294,607; 5,371,108; and 5,578,586; all of which are incorporated herein by reference. Lubricating compositions based on liposomes are described in U.S. Pat. No. 4,818,537 and U.S. Pat. No. 5,800,807, the disclosures of which are incorporated by reference herein.

In some embodiments, the additive can be one or more tonicity agents, which can be used to adjust the tonicity of the composition, for example, to the tonicity of natural tears. Suitable tonicity agents include, by way of example and not limitation, dextrans (e.g., dextran 40 or 70), dextrose, glycerin, potassium chloride, propylene glycol, and sodium chloride. Equivalent amounts of one or more salts made up of cations, for example, such as potassium, ammonium and anions such as chloride, citrate, ascorbate, borate, phosphate, bicarbonate, sulfate, thiosulfate, bisulfate; the salts sodium bisulfate and ammonium sulfate can also be used. The amount of tonicity agent will vary, depending on the particular agent to be added. In general, however, the compositions will have a tonicity agent in an amount sufficient to cause the final composition to have an ophthalmically acceptable osmolarity, for example, about 250 to about 450 mOsM/L, or about 250 to about 350 mOsM/L, as discussed above.

In some embodiments, the ocular solution is a composition of NaMPA and a preservative. In particular, the preservative is EDTA, which can be present from about 0.005 to about 0.050% w/v, about 0.005 to about 0.040% w/v, about 0.010 to about 0.030% w/v, about 0.010 to about 0.020% w/v, or from about 0.010 to about 0.015% w/v. In some embodiments, the EDTA is present at about 0.005, 0.01, 0.012, 0.014, 0.016, 0.018, 0.020, 0.030, 0.040, or 0.050% w/v. In some embodiments, the EDTA (as disodium dehydrate) is present at about 0.012% w/v.

In some embodiments, the ocular solution includes NaMPA, a preservative, and a buffering agent. An exemplary formulation of this type can include the preservative EDTA, in amounts as noted above; a buffering agent, such as borate or tromethamine, in an amount of that provides a buffering capacity of 0.01 to about 0.1; and a solution pH of about 7.0 to 8.0.

In the embodiments herein, the ocular solution can be formulated in accordance with methods known in the art. Guidance can be found in Duvall and Kershner, Ophthalmic Medications and Pharmacology 2^{nd} Ed, Slack Incorporated (2006); Ophthalmic Drug Facts® 18^{th} Ed, Wolters Kluwer (2007); Remington's Pharmaceutical Sciences, 19th ed. Gennaro AR, ed. Easton, Pa.: Mack Publishing, pages 1581-1959 (1990); and Reynolds La., 1991, “Guidelines for preparation of sterile ophthalmic products,” Am J Hosp Pharm. 48:2438-9; the disclosures of which are incorporated by reference herein.

Given the ability of the NaMPA in the formulation to penetrate the eye, the ocular solutions described herein can be used to treat various ocular disorders amenable to treatment with the immunosuppressive and anti-inflammatory compound. The terms “ophthalmic disorder,” “ocular disorder,” “ocular disease,” and “eye disorder” are used interchangeably herein to include, among others, “back-of-eye” diseases involving the retina, macula, fovea, etc. in the posterior region of the eye; and “front-of-eye” diseases, such as those that involve tissues such as the cornea, iris, ciliary body, conjunctiva, lacrimal gland, etc. These conditions or diseases can manifest as pain, discomfort, tissue damage and compromised visual performance of the eyes in the afflicted subject.

Examples of “back-of-eye” disease include, among others, macular edema such as angiographic cystoid macular edema; retinal ischemia and choroidal neovascularization; macular degeneration; retinal diseases (e.g., diabetic retinopathy, diabetic retinal edema, retinal detachment); inflammatory diseases such as uveitis (including panuveitis) or choroiditis (including multifocal choroiditis) of unknown cause (idiopathic) or associated with a systemic (e.g., autoimmune) disease; episcleritis or scleritis; Birdshot retinochoroidopathy; vascular diseases (retinal ischemia, retinal vasculitis, choroidal vascular insufficiency, choroidal thrombosis); neovascularization of the optic nerve; and optic neuritis.

Examples of “front-of-eye” diseases include, among others, blepharitis; keratitis; rubeosis iritis; Fuchs' heterochromic iridocyclitis; chronic uveitis or anterior uveitis; conjunctivitis; allergic conjunctivitis (including seasonal or perennial, vernal, atopic, and giant papillary); keratoconjunctivitis sicca (dry eye syndrome); iridocyclitis; iritis; scleritis; episcleritis; corneal edema; scleral disease; ocular cicatrcial pemphigoid; pars planitis; Posner Schlossman syndrome; Behçet's disease; Vogt-Koyanagi-Harada syndrome; hypersensitivity reactions; conjunctival edema; conjunctival venous congestion; periorbital cellulitis; acute dacryocystitis; non-specific vasculitis; and sarcoidosis.

In some embodiments, the eye disorder is associated with an inflammatory condition of the eye. These conditions can include, but are not limited to, the various disorders described above for the back-of-eye and front-of-eye, such as, for example, inflammation associated with macular edema; retinal ischemia; choroidal neovascularization, macular degeneration; diabetic retinopathy; diabetic retinal edema; retinal detachment; inflammatory diseases such as uveitis (including panuveitis) or choroiditis (including multifocal choroiditis) of unknown cause (idiopathic) or associated with a systemic (e.g., autoimmune) disease; episcleritis or scleritis; Birdshot retinochoroidopathy; vascular diseases (retinal ischemia, retinal vasculitis, choroidal vascular insufficiency, choroidal thrombosis); neovascularization of the optic nerve and optic neuritis; blepharitis; keratitis; rubeosis iritis; Fuchs' heterochromic iridocyclitis; chronic uveitis or anterior uveitis; conjunctivitis; allergic conjunctivitis (including seasonal or perennial, vernal, atopic, and giant papillary); keratoconjunctivitis sicca (dry eye syndrome); iridocyclitis; iritis; scleritis; episcleritis; corneal edema; scleral disease; ocular cicatrcial pemphigoid; pars planitis; Posner Schlossman syndrome; Behçet's disease; Vogt-Koyanagi-Harada syndrome; hypersensitivity reactions; conjunctival edema; conjunctival venous congestion; periorbital cellulitis; acute dacryocystitis; non-specific vasculitis; and sarcoidosis.

In some embodiments, the eye disorder treatable with the ocular formulation is keratoconjunctivitis sicca, a condition also known as dry-eye, keratitis sicca, sicca syndrome, xeropthalmia, and dry eye syndrome (DES), which can arise from decreased tear production and/or increased tear film evaporation due to abnormal tear composition. Although the disorder can be caused by environmental chemicals and infection, the disorder is also associated with the autoimmune diseases rheumatoid arthritis, lupus erythematosus, diabetes mellitus, and Sjögren's syndrome.

In some embodiments, the eye disorders that can be treated with the formulations are those associated with autoimmune disorders. These conditions can include, but are not limited to, the various disorders above described for the back-of-eye and front-of-eye, such as, for example, choroidal neovascularization; macular degeneration; diabetic retinopathy; diabetic retinal edema; uveitis (including panuveitis) or choroiditis (including multifocal choroiditis) of unknown cause (idiopathic) or associated with a systemic disorder (e.g., autoimmune disease); episcleritis or scleritis; Birdshot retinochoroidopathy; neovascularization of the optic nerve, and optic neuritis; blepharitis, keratitis, rubeosis iritis; Fuchs' heterochromic iridocyclitis; chronic uveitis or anterior uveitis; conjunctivitis; allergic conjunctivitis (including seasonal or perennial, vernal, atopic, and giant papillary); iridocyclitis; iritis; scleritis; episcleritis; corneal edema; scleral disease; ocular cicatrcial pemphigoid; pars planitis; Posner Schlossman syndrome; Behçet's disease; Vogt-Koyanagi-Harada syndrome; hypersensitivity reactions; conjunctival edema; conjunctival venous congestion; periorbital cellulitis; acute dacryocystitis; non-specific vasculitis; and sarcoidosis.

In some embodiments, the eye disorder associated with an autoimmune condition that can be treated with the ocular formulations is uveitis, a general term used to describe inflammation of any component of the uveal tract. The uveal tract of the eye consists of the iris, ciliary body, and choroid. Inflammation of the underlying retina, called retinitis, or of the optic nerve, called optic neuritis, or overlying sclera called scleritis or episcleritis may occur with or without accompanying uveitis. Uveitis can be classified based on the segment of the eye that is affected, such as anterior, intermediate, posterior, or diffuse, or on the specific anatomical part involved, such as iritis, iridocyclitis, or choroiditis. Posterior uveitis signifies any of a number of forms of retinitis, choroiditis, or optic neuritis, as further described below. Diffuse uveitis typically implicates inflammation involving all parts of the eye, including anterior, intermediate, and posterior structures. Uveitis is one of the most common ocular disorders associated with autoimmune diseases, including rheumatoid arthritis; systemic lupus erythematosus; Sjogren's syndrome; diabetes mellitus; sarcoidosis; ankylosing spondylitis; Psoriasis; multiple sclerosis; Vogt-Koyanagi-Harada disease; Behçet's disease; polyarteris nodosa; giant cell arteritis; and inflammatory bowel disease.

In some embodiments, inflammatory eye conditions such as conjunctivitis, blepharitis; keratitis; vitirits; chorioretinitis; and uveitis is associated with systemic or local infections where an immunosuppressant drug such as NaMPA may be used topically to suppress the ocular inflammation. Infections may be due to bacterial (e.g., Borrelia species, Streptococcus pneumoniae, Staphylococcus aureus, Mycobacterium tuberculosis, Mycobacterium leprae, Neisseria gonorrheae, Chlamydia trachomatis, Pseudomonas aeruginosa, etc.), viral (e.g., Herpes simplex, Herpes zoster, cytomegalovirus, etc.), fungal (e.g., Aspergillus fumigatus, Candida albicans, Histoplasmosis capsulatum, Cryptococcus species, Pneumocystis carinii, etc.) or parasitic agents (e.g., Toxoplasmosis gondii, Trypanosome cruzi, Leishmania species, Acanthamoeba species, Giardia lamblia, Septata species, Dirofilaria immitis, etc.).

In some embodiments, the ocular solutions will generally be used in an amount effective to treat the particular ocular disorder or disease in a subject in need thereof. The ocular solutions may be administered therapeutically to achieve therapeutic benefit or prophylactically to achieve prophylactic benefit. As used herein, a “subject” is generally any animal that may benefit from administration of the therapeutic agents described herein. The therapeutic agents may be administered to a mammalian subject, such as a human subject. In some embodiments, the therapeutic agents may be administered to a veterinary animal subject, such as, among others, mouse, rat, horse, cat, dog, cow, pig, monkey, chimpanzee, etc.

By “treating” or “treatment” is meant medically managing a subject (e.g., a patient) with the intent that a prevention, cure, stabilization, or amelioration of the symptoms will result. Treatment includes active treatment, that is, treatment directed specifically towards improvement of the disease; palliative treatment, that is, treatment designed for the relief of symptoms rather than the curing of the disease; preventive treatment, that is, treatment directed to prevention of the disease; and supportive treatment, that is, treatment employed to supplement another specific therapy directed toward the improvement of the disease. As such, “treatment” also refers to delaying the onset of the disease or disorder, or inhibiting the disease or disorder, thereby providing a prophylactic benefit.

In the embodiments herein, a therapeutically effective amount is applied topically to the eye of a subject in need of treatment. A “therapeutically effective amount” refers to an amount of the therapeutic agent either as an individual compound or in combination with other compounds that is sufficient to induce a therapeutic effect or prophylactic benefit on the disease or condition being treated. This phrase should not be understood to mean that the dose must completely eradicate the ailment. A therapeutically effective amount will vary depending on, inter alia, the pharmacological properties of the compound used in the methods, the condition being treated, the frequency of administration, the mode of delivery, characteristics of the individual to be treated, the severity of the disease, and the response of the patient. A skilled artisan can take into account such factors when formulating compositions for the treatments described herein, a process which is well within the skill of those in the art.

To treat the ocular disease, the ophthalmic compositions can be applied topically to the affected eye(s). In some embodiments, the ocular formulation can be applied in defined volumes, such about 10, 20, 50, 75, 100, 150, or 200 μl or more. The frequency of application will depend on, among others, the type of ocular disease being treated, the severity of the condition, age and sex of the patient, the amount of the NaMPA in the formulation, and the pharmacokinetic profile in the ocular tissue to be treated. In some embodiments, the ocular solution can be administered more than one times per day. When the compositions are administered more than once per day, the frequency of administration can be two, three, four, up to eight times per day. In some embodiments, the ocular solution can be administered one to four times daily. In some embodiments, the ocular solution can be applied once every two days. In some embodiments, the ocular formulation can be applied once every four days. In some embodiments, the ocular formulation can be administered once every week. Determining the frequency and amount to be administered for a particular ocular disorder is well within the skill and judgment of the attending practitioner.

In some embodiments, the ocular formulation can be provided in the form of a kit. As such, the kit can contain the ocular formulation in a container, as single dose unit or as a single solution reservoir. The kit can also contain a dispenser for dispensing measured doses as well as instructions for dosing and use of the formulations.

Having now generally described the invention, the same will be more readily understood through reference to the following examples which are provided by way of illustration, and are not intended to be limiting of the present invention, unless specified.

### 6. EXAMPLES

### Example 1

### Preparation of Ocular Solutions of NaMPA

Ophthalmic Solution 1.4% MPA Ophthalmic Solution, Sodium Salt

Ophthalmic Solution 1

| Ingredients | % (W/V) |
|---|---|
| Mycophenolic Acid | 4.0 |
| Glycerin, USP | 0.8 |
| NaOH, NF | ~0.64 |
| NaOH/HCl, NF | pH 7.2-7.6 |
| Purified Water, USP | q.s 100 |

Preparation procedure. Purified water, about 80% of final volume, was heated to approximately 80° C. Glycerin and mycophenolic acid was added to the water and mixed to disperse. Heating was stopped and NaOH 10% added immediately to the batch and mixed until MPA was dissolved. Alternatively, purified water (about 80% of final volume), NaOH 10%, and glycerin were mixed and heated to approximately 80° C. Heating was stopped and then mycophenolic acid added, and mixed to dissolve. Purified water was added to approximately 95% of batch volume, and the composition mixed while cooling to room temperature. The pH was measured with a calibrated pH meter and the pH adjusted with NaOH/HCl as necessary. Purified water was added to 100% of batch volume and the osmolarity measured. Appropriate filters were used for clarification and sterilization.

Ophthalmic Solution 2: 4% MPA Ophthalmic Solution, Tromethamine Salt

Ophthalmic Solution 2

| Ingredients | % (w/v) |
|---|---|
| Mycophenolic Acid | 4.0 |
| Glycerin, USP | 0.72 |
| Tromethamine, USP | ~2.12 |
| NaOH/HCl, NF | pH 7.2-7.6 |
| Purified Water, USP | q.s 100 |

Preparation procedure. Purified water, about 80% of final volume, was heated to approximately 80° C. Glycerin and mycophenolic acid was added to the water and mixed to disperse. Heating was stopped, and tromethamine was immediately added to the batch and mixed until MPA dissolved. Alternatively, purified water, about 80% of final volume, tromethamine and glycerin were mixed and heated to approximately 80° C. Heating was stopped and then mycophenolic acid added, and the solution mixed to dissolve the MPA. Purified water was added to approximately 95% of batch volume, and the solution mixed while cooling to room temperature. The pH was measured with a calibrated pH meter and if necessary, the pH adjusted with NaOH/HCl. Purified water was added to 100% of batch volume and the osmolarity measured. Filters were used for clarification and sterilization, where appropriate.

Ophthalmic Solution 3: 3% MPA Ophthalmic Solution, (Hydroxyethyl morpholine Salt).

Ophthalmic Solution 3

| Ingredients | % (w/v) |
|---|---|
| Mycophenolic Acid | 3.0 |
| Glycerin, USP | 0.49 |
| Hydroxyethyl morpholine | ~2.28 |
| NaOH/HCl, NF | pH 7.2-7.6 |
| Purified Water, USP | q.s 100 |

Preparation procedure. Purified water, about 80% of final volume, was heated to approximately 80° C. Glycerin and mycophenolic acid was added to water and mixed to disperse. Heating was stopped and hydroxyethylmorpholine immediately added to the batch and mixed until MPA dissolved. Alternatively, purified water (about 80% of final volume), hydroxyethylmorpholine and glycerin were mixed and heated to approximately 80° C. Heating was stopped and mycophenolic acid added, and the solution mixed to dissolve the MPA. Purified water was added to approximately 95% of batch volume, and the solution mixed while cooling to room temperature. The pH was measured with a calibrated pH meter and if necessary, the pH adjusted with NaOH/HCl. Purified water was added to 100% of batch volume and the osmolarity measured. Appropriate filters were used for clarification and sterilization.

### Example 2

### Ophthalmic Formulations with EDTA

The table below provides various ophthalmic formulations of NaMPA with additive EDTA and various levels of NaCl.

TABLE 1
Ophthalmic Solutions with EDTA

| | Formulation | | | | | |
|---|---|---|---|---|---|---|
| Ingredients | 1 | 2 | 3 | 4 | 5 | 6 |
| NaMPA | 4.275 | 2.14 | 1.070 | 0.535 | 0.267 | 0 |
| (MPA) | (4.0) | (2.0) | (1.0) | (0.5) | (0.25) | (0.0) |
| % w/v | | | | | | |
| NaCl | 0.34 | 0.60 | 0.72 | 0.80 | 0.82 | 0.85 |
| % w/v | | | | | | |
| Edetate | 0.012 | 0.012 | 0.012 | 0.012 | 0.012 | 0.012 |
| Disodium | | | | | | |
| (2H₂O) | | | | | | |
| % w/v | | | | | | |
| NaOH/HCl | pH | pH | pH | pH | pH | pH |
| | 7.5 ± | 7.5 ± | 7.5 ± | 7.5 ± | 7.5 ± 0.2 | 7.0-8.0 |
| | 0.2 | 0.2 | 0.2 | 0.2 | | |
| Purified | q.s 100 | q.s 100 | q.s 100 | q.s 100 | q.s 100 | q.s 100 |
| Water | | | | | | |

The above Table shows ophthalmic NaMPA formulations used in animal studies, including the efficacy studies described below. The first three ingredients, NaMPA, NaCl and edetate (EDTA) disodium, are shown as final % concentrations in weight per volume (w/v). Formulations are brought to the desired pH as shown using NaOH or HCl, and brought to a final volume of 100 ml with purified water. Other volumes of ophthalmic NaMPA formulations can be made using the formulas outlined in Table 1. Under “NaMPA”, the final concentration of MPA is shown in parentheses. For example, formulation 3 refers to a 1% NaMPA formulation. A 1% NaMPA formulation contains a final concentration of 1% MPA, the active ingredients.

The solutions above were made by the methods described and tested for tolerability, tissue penetration, and efficacy, as further described below.

### Example 3

### Studies on Tolerability and Ocular Tissue Penetration of MPA Containing Formulations as Compared to Cyclosporine

The objective of this study was to evaluate the ocular tolerability and ocular tissue penetration of eight MPA formulations following topical instillation into the eyes of New Zealand White rabbits eight times a day for one day. Eight test article formulations, a negative control article (2.4% glycerin), and a positive control article (Restasis®, manufactured by Allergan Inc. (Allergan) of Irvine, Calif., US) are provided in Table 2. The study was conducted in two phases, with four test articles and both control articles used in each phase. Naïve animals were used in both phases. Animals were placed into treatment groups as noted in Tables 2, 3 and 4.

TABLE 2
Treatment Group Assignment: Phase 1

| | | | | | Dose |
|---|---|---|---|---|---|
| | | Ocular Treatment | | | (each |
| Group | No. | (Both Eyes) | Frequency | Route | eye) |
| A | 2 | Negative Control (2.4% | 8x Daily | Topical | 40 μL |
| | | Glycerin) | | Instillation | |
| B | 2 | 1% MPA - sodium salt | 8x Daily | Topical | 40 μL |
| | | | | Instillation | |
| C | 2 | 1% MPA - sodium salt + | 8x Daily | Topical | 40 μL |
| | | borate | | Instillation | |
| D | 2 | 1% MPA - tromethamine | 8x Daily | Topical | 40 μL |
| | | salt | | Instillation | |
| E | 2 | 1% MPA | 8x Daily | Topical | 40 μL |
| | | hydroxyethylmorpholine | | Instillation | |
| | | salt | | | |
| F | 2 | Positive Control | 8x Daily | Topical | 40 μL |
| | | (Restasis ®) | | Instillation | |

MPA = mycophenolic acid.

TABLE 3
Treatment Group Assignment: Phase 2

| | | | | | Dose |
|---|---|---|---|---|---|
| | | Ocular Treatment | | | (each |
| Group | No. | (Both Eyes) | Frequency | Route | eye) |
| G | 2 | Negative Control (2.4% | 8x Daily | Topical | 40 μL |
| | | Glycerin) | | Instillation | |
| H | 2 | 2% MPA - sodium salt | 8x Daily | Topical | 40 μL |
| | | | | Instillation | |
| I | 2 | 2% MPA - sodium | 8x Daily | Topical | 40 μL |
| | | salt + borate | | Instillation | |
| J | 2 | 2% MPA - tromethamine | 8x Daily | Topical | 40 μL |
| | | salt | | Instillation | |
| K | 2 | 2% MPA | 8x Daily | Topical | 40 μL |
| | | hydroxyethylmorpholine | | Instillation | |
| | | salt | | | |
| L | 2 | Positive Control | 8x Daily | Topical | 40 μL |
| | | (Restasis ®) | | Instillation | |

MPA = mycophenolic acid.

TABLE 4
Treatment Group Assignment: Phase 3

| | | | | | Dose |
|---|---|---|---|---|---|
| | | Ocular Treatment | | | (each |
| Group | No. | (Both Eyes) | Frequency | Route | eye) |
| A | 2 | Negative Control (2.4% | 8x Daily | Topical | 40 μL |
| | | Glycerin) | | Instillation | |
| B | 2 | 4% MPA - sodium salt | 8x Daily | Topical | 40 μL |
| | | | | Instillation | |
| C | 2 | 4% MPA - sodium | 8x Daily | Topical | 40 μL |
| | | salt + borate | | Instillation | |
| D | 2 | 4% MPA - tromethamine | 8x Daily | Topical | 40 μL |
| | | salt | | Instillation | |
| E | 2 | 3% MPA | 8x Daily | Topical | 40 μL |
| | | hydroxyethylmorpholine | | Instillation | |
| | | salt | | | |
| F | 2 | Positive Control | 8x Daily | Topical | 40 μL |
| | | (Restasis ®) | | Instillation | |

MPA = mycophenolic acid.

Phase 1 and Phase 2 Studies

Twenty-four female New Zealand White rabbits were obtained from The Rabbit Source (Ramona, Calif., US). Animals were 15-19 weeks old and weighed 2.16-3.23 kg on Day 1. The protocol specified that study animals would weigh 1.5-2.5 kg on Day 1, but eight phase 1 animals and all phase 2 animals weighed 0.06-0.73 kg more than the specified maximum. This deviation was not believed to have an effect on the outcome of the study. Animals were identified by ear tags and cage cards.

Quarantine and care of animals were performed under the standard operating procedure for the laboratory (SOP). Upon arrival, animals were quarantined for 8-10 days and examined to ensure they were in good health. Housing, sanitation, and environmental monitoring were performed under an SOP. The animals were housed in individual, hanging, stainless steel cages. The study room temperature was 72-74° F. with 30-48% relative humidity. Animals received Teklad Certified Hi Fiber Rabbit Diet daily and tap water ad libitum.

Prior to placement on study, both eyes of each animal were grossly evaluated for signs of irritation or discomfort and observations were scored according to an SOP and recorded using a standardized data collection sheet. No rabbits with gross signs of ocular irritation were used in this study.

Prior to placement on study, each animal underwent a pre-treatment ophthalmic examination (slit lamp with fluorescein). Ocular findings were scored according to an SOP and recorded using a standardized data collection sheet. Acceptance criteria for placement on study were as follows: scores of ≦1 for conjunctival congestion and swelling; and scores of 0 for all other observation variables. Eyes were re-evaluated by slit lamp opthalmoscopy with fluorescein immediately prior to dosing on Day 1. Animals with any ocular abnormalities immediately prior to dosing were replaced.

Prior to dosing in each phase, 12 animals were weighed and randomly assigned to six study groups. Each randomization was based on a modified Latin square. Dosing was performed on Day 1 of each phase as follows: The test and control articles were brought to room temperature prior to dosing. At the appropriate intervals as specified in the treatment group tables, 40 μL of test or control article was administered using a calibrated pipette into both eyes of each animal. Animals were dosed eight times with doses administered at target intervals of 1 hour ±5 minutes apart. Following each dose, eyelids were held closed for 10 seconds. The time of each dose administration was recorded.

Both eyes of each animal were grossly evaluated for signs of irritation or discomfort prior to the first dose on Day 1 and at target intervals of 15-30 minutes following each successive dose. Gross observations were scored and recorded using a standardized data collection sheet. Signs of discomfort observed immediately after each dose were noted in the study records.

Ophthalmic examinations (slit lamp with fluorescein) were performed on both eyes of each animal on Day 1 (prior to the first dose and following the last dose). Ocular findings were scored according to an SOP and recorded using a standardized data collection sheet.

Animals were observed for mortality/morbidity twice daily. Animals were weighed at randomization and on Day 1 (prior to the first dose).

One animal from each of the groups was randomly selected for ocular tissue collection. The selected animals were euthanized with an intravenous injection of commercial euthanasia solution following final ophthalmic examinations. Euthanasia was performed according to an SOP. Remaining animals were returned to the vivarium for possible additional phases of the study.

Ocular tissues were collected from each euthanized animal as follows: The aqueous humor was collected from each eye, and the volume of aqueous humor was measured. Both globes and surrounding tissues, including lacrimal glands and eyelids, were collected. The lacrimal glands and eyelids were weighed as a single complex. The conjunctiva was collected from each globe and weighed. All collected tissues were snap-frozen in liquid nitrogen. After freezing, the following tissues were collected from the eyes of the test animals: cornea, iris-cilliary body complex, lens, vitreous humor, choroid-retina complex, and sclera. Tissues were collected according to SOP. Ocular tissues dissected from Group B-E and H-K globes were weighed, labeled, and stored frozen (−70° C.). All ocular tissues of Group B-E and H K animals were then shipped on dry ice to IAS for analysis of MPA concentrations. Ocular tissues of Group F and L animals (Restasis® dose groups) were stored frozen (−70° C.) at the BTC.

Phase 3 Studies

Methods were similar to those presented above for phase 1 and phase 2. One animal each from Groups B-F was randomly selected for ocular tissue collection. The selected animals were euthanized with an intravenous injection of commercial euthanasia solution following final ophthalmic examinations. Euthanasia was performed according to a SOP. Remaining animals were returned to the vivarium for additional phases of the study.

Ocular tissues were collected from each euthanized animal as follows: the aqueous humor was collected from each eye, and the volume of aqueous humor was measured. Both globes and surrounding tissues, including lacrimal glands and eyelids, were collected. The lacrimal glands and eyelids were weighed separately. The conjunctiva was collected from each globe and weighed. All collected tissues were snap-frozen in liquid nitrogen. After freezing, the following tissues were collected from eyes of Group B-E animals: cornea, iris-cilliary body complex, lens, vitreous humor, choroid retina complex, and sclera. Tissues were collected according to an SOP. Collected ocular tissues were weighed, labeled, and stored frozen (−70° C.) until shipped on dry ice for analysis of MPA concentrations.

Results of Phase 1 and Phase 2 Studies

Eyes dosed with 1% MPA hydroxyethylmorpholine salt formulation (phase 1) had hyperemia and chemosis that was visible at gross and ophthalmic examinations. The hyperemia and chemosis in these eyes were similar to the irritation seen in eyes dosed with Restasis®. Eyes dosed with 2% MPA hydroxyethylmorpholine salt formulation (phase 2) showed marked acute discomfort immediately after dose administrations. However, these eyes appeared normal at gross and ophthalmic examinations, with no hyperemia or congestion.

Results of Phase 3 Studies

All formulations were well tolerated according to gross observations made post-application, with the exception of the 3% MPA hydroxyethylmorpholine salt solution (Group E). Both Group E animals exhibited signs of moderate ocular discomfort (blinking, keeping eyes shut, and pawing at eyes) immediately after instillations of the test article. The left eye of Group E Rabbit No. 1650 showed signs of ocular irritation (hyperemia, chemosis, and discharge) at gross observations following the last three test article instillations. The same eye developed a geographic superficial corneal ulcer by the end of the study. The lesion was consistent with epithelial toxicity associated with drug administration.

MPA Levels in Ocular Tissues

As noted above, one animal from the groups receiving MPA or cyclosporine was randomly selected for ocular tissue collection. Ocular tissues collected were aqueous humor, conjunctiva, cornea, eye lid, iris-ciliary body, lacrimal gland, lens, retina/choroid, sclera and vitreous humor. Lacrimal glands were collected only from cyclosporine-treated animals in phase 1 and phase 2, and lenses were collected only from MPA treated animals. Selected animals were euthanized with an intravenous injection of commercial euthanasia solution following final ophthalmic examinations, which would be approximately 1 hr following the eighth dose.

In general, the following observations were made: (1) MPA concentrations were higher in anterior, external ocular tissues and lower in posterior, intraocular tissues; (2) there were no major differences in ocular bioavailability between formulations; and (3) increasing concentration of MPA in the applied solution increased the concentration of MPA found in some tissues, but not others.

Mean tissue concentrations of MPA in one external ocular tissue (cornea) and one intraocular tissue (iris-ciliary body) are shown in Table 5. Mean MPA concentrations were approximately 20-70 mcg/g (6.4-22.4 μM) in the cornea, and 0.50 5.0 mcg/g (0.16 1.6 μM) in the iris/ciliary-body.

TABLE 5
MPA Concentrations in Selected Ocular Tissues (ng/g)

| Matrix | Phase | Group | N | Mean | SD |
|---|---|---|---|---|---|
| Cornea | P1 | 1% MPA HEM | 2 | 17550 | 636 |
| | | 1% MPA Na | 2 | 20100 | 3818 |
| | | 1% MPA Na&Bo | 2 | 20350 | 2899 |
| | | 1% MPA Tro | 2 | 14475 | 7955 |
| | | Cyclosporine | 2 | 115 | 72 |
| | P2 | 2% MPA HEM | 2 | 34700 | 3111 |
| | | 2% MPA Na | 2 | 50950 | 6435 |
| | | 2% MPA Na&Bo | 2 | 65050 | 23971 |
| | | 2% MPA Tro | 2 | 71750 | 33022 |
| | | Cyclosporine | 2 | 343 | 88 |
| | P3 | 3% MPA HEM | 2 | 33100 | 1838 |
| | | 4% MPA Na | 2 | 50850 | 3323 |
| | | 4% MPA Na&Bo | 2 | 43450 | 4172 |
| | | 4% MPA Tro | 2 | 55950 | 26941 |
| | | Cyclosporine | 2 | 297 | 88 |
| Iris/Ciliary | P1 | 1% MPA HEM | 2 | 608 | 373 |
| body | | 1% MPA Na | 2 | 2155 | 728 |
| | | 1% MPA Na&Bo | 2 | 872 | 464 |
| | | 1% MPA Tro | 2 | 417 | 275 |
| | | Cyclosporine | 2 | 6 | 2 |
| | P2 | 2% MPA HEM | 2 | 1420 | 368 |
| | | 2% MPA Na | 2 | 1830 | 1160 |
| | | 2% MPA Na&Bo | 2 | 1519 | 738 |
| | | 2% MPA Tro | 2 | 1322 | 719 |
| | | Cyclosporine | 2 | 5 | 3 |
| | P3 | 3% MPA HEM | 2 | 3625 | 771 |
| | | 4% MPA Na | 2 | 4680 | 1711 |
| | | 4% MPA Na&Bo | 2 | 5415 | 983 |
| | | 4% MPA Tro | 2 | 4925 | 1209 |
| | | Cyclosporine | 2 | 193 | 140 |

HEM = hydroxyethylmorpholine salt;
MPA = mycophenolic acid;
Na = sodium salt;
Na&Bo = sodium salt + borate;
Tro = tromethamine salt.

### Example 4

### Studies on Penetration of NaMPA Containing Formulations as Compared to Cyclosporine

The objective of this study was to evaluate the pharmacokinetics and ocular toxicity of three test article formulations following topical instillation into the eyes of New Zealand White rabbits at a frequency of 2, 4, or 8 times per day for 14 days. Three different test article formulations, a negative control article (2.4% glycerin), and a positive control article (Restasis®, manufactured by Allergan Inc., Irvine, Calif., US) are provided in Table 6.

TABLE 6
Mycophenolic Acid Ophthalmic Solution: Test Articles

| Test or Control Article | Formulation No. | Lot No. |
|---|---|---|
| 4% MPA - sodium salt (in glycerin, | N189 | NRI 336 |
| purified water) | | |
| 4% MPA - tromethamine salt (in glycerin, | N190 | NRI 337 |
| purified water) | | |
| 3% MPA - hydroxyethylmorpholine salt | N191 | NRI 338 |
| (in glycerin, purified water) | | |
| Negative control (2.4% glycerin) | N192 | NRI 339 |
| Positive control (Restasis ®, cyclosporine | N/A | Allergan |
| ophthalmic emulsion 0.05%) | | 46845 |

MPA = mycophenolic acid;
NRI = Newport Research, Inc.;
N/A = not applicable.

Animals were placed into treatment groups as noted in Table 7.

TABLE 7
Treatment Group Assignment

| | | Ocular Treatment | | | Dose | |
|---|---|---|---|---|---|---|
| Group | No. | (Both Eyes) | Frequency | Route | (Each Eye) | Phase 1 |
| A | 4 | Negative control (2.4% | 8x Daily | Topical | 40 μL | 2 |
| | | Glycerin) | | Instillation | | |
| B | 4 | 4% MPA - sodium salt | 2x Daily | Topical | 40 μL | 1 |
| | | | | Instillation | | |
| C | 4 | 4% MPA - sodium salt | 4x Daily | Topical | 40 μL | 1 |
| | | | | Instillation | | |
| D | 4 | 4% MPA - sodium salt | 8x Daily | Topical | 40 μL | 1 |
| | | | | Instillation | | |
| E | 4 | 4% MPA - tromethamine | 2x Daily | Topical | 40 μL | 1 |
| | | salt | | Instillation | | |
| F | 4 | 4% MPA - tromethamine | 4x Daily | Topical | 40 μL | 1 |
| | | salt | | Instillation | | |
| G | 4 | 4% MPA - tromethamine | 8x Daily | Topical | 40 μL | 1 |
| | | salt | | Instillation | | |
| H | 4 | 3% MPA - | 2x Daily | Topical | 40 μL | 2 |
| | | hydroxyethylmorpholine | | Instillation | | |
| | | salt | | | | |
| I | 4 | 3% MPA - | 4x Daily | Topical | 40 μL | 2 |
| | | hydroxyethylmorpholine | | Instillation | | |
| | | salt | | | | |
| J | 4 | 3% MPA - | 8x Daily | Topical | 40 μL | 2 |
| | | hydroxyethylmorpholine | | Instillation | | |
| | | salt | | | | |
| K | 4 | Positive control (Restasis ®) | 8x Daily | Topical | 40 μL | 2 |
| | | | | Instillation | | |

MPA = mycophenolic acid.

The test articles and negative control were stored at room temperature for four days and then moved to refrigerated storage (2-8° C.). The positive control was stored at room temperature throughout the study.

Forty-four female New Zealand White rabbits were obtained from The Rabbit Source (Ramona, Calif., US). Animals were 13-21 weeks old and weighed 1.81-2.90 kg on Day 1. Quarantine and care of animals were performed per a Biological Control Operating Procedure (BCOP). Upon arrival, animals were quarantined for 10 days and examined to ensure they were in good health. Housing, sanitation, and environmental monitoring were performed per BCOP. The animals were housed in individual, hanging, stainless steel cages. The study room temperature was 63-76° F. with 40-70% relative humidity. Animals received Teklad Certified Hi Fiber Rabbit Diet daily and tap water ad libitum.

Prior to placement on study, both eyes of each animal were grossly evaluated for signs of irritation or discomfort. Observations were scored according to a laboratory standard operating procedure (SOP) and recorded using a standardized data collection sheet. Prior to placement on study, each animal underwent a pre-treatment ophthalmic examination (slit-lamp with fluorescein). Ocular findings were scored according to an SOP and recorded using a standardized data collection sheet. Acceptance criteria for placement on study were as follows: scores of ≦1 for conjunctival congestion and swelling; scores of 0 for all other observation variables. Eyes were re evaluated by slit-lamp opthalmoscopy with fluorescein immediately prior to dosing on Day 1.

Prior to dosing, 44 animals were weighed and randomly assigned to 11 study groups. Randomization was based on a modified Latin square. Treatment groups are shown in Table 7.

The study was conducted in two phases, with Groups B-G treated in phase 1 and Groups A and H-K treated in phase 2. Fifteen original study animals (five in the phase 1 group and 10 in the phase 2 group) were replaced due to conjunctival congestion that developed after randomization. All animals used in phase 2 were weighed and re randomized to groups immediately prior to phase 2 dosing.

Dosing was performed on Days 1-14 of each phase as follows: at the appropriate intervals as specified in the treatment group table, 40 μL of test or control article was administered using a calibrated pipette into both eyes of each animal. Following each dose, eyelids were held closed for 10 seconds. The time of each dose administration was recorded.

Doses were administered twice daily (7 or 8 hours apart), four times daily (2 hours apart), or eight times daily (1 hour apart). The protocol specified that doses would be administered twice daily (8 hours±5 minutes apart), four times daily (2 hours±5 minutes apart), or eight times daily (1 hour±5 minutes apart). In phase 1, all doses were administered within the specified time intervals. In phase 2, doses were administered outside of the specified intervals as follows: on Days 1-14, the second dose was given 7 hours ±5 minutes after the first dose to all Group H eyes. On Day 6, the sixth dose was given 1-3 minutes late to 17 eyes (Group A, J, or K). On Day 8, the second dose was given 1 minute late to 2 eyes, fourth dose was given 3 minutes late to 8 eyes (Group I), the sixth dose was given 1-2 minutes late to 16 eyes (Group J or K), the seventh dose was given 1-4 minutes late to 18 eyes (Group A, J, or K), and the eighth dose was given 17 minutes late to 20 eyes (Group A, J, or K). On Day 9, the fourth dose was given 14 minutes late to 8 eyes (Group I), the sixth dose was given 1-2 minutes late to 6 eyes (Group K), the seventh dose was given 1-3 minutes late to 10 eyes (Group J or K), and the eighth dose was given 1-2 minutes late to 14 eyes (Group J or K). On Day 14, the second dose was given one minute early to 8 eyes (Group I), and the third dose was given one minute early to 14 eyes (Group A or K). These deviations in dosing intervals are believed to have minimal effect on the outcome of the study.

On Days 1-14, both eyes of each animal were grossly evaluated for signs of irritation or discomfort before the first dose of the day and 15-30 minutes after the last dose of the day. Immediately after each dose administration, signs of ocular discomfort and duration were recorded. Gross observations were scored according to an SOP and recorded using a standardized data collection sheet.

Ophthalmic examinations (slit-lamp with fluorescein) were performed on all eyes prior to the first dose on Day 1 and immediately after gross ocular observations on Days 7 and 14. Ocular findings were scored according to an SOP and recorded using a standardized data collection sheet.

Animals were observed for mortality/morbidity twice daily. Animals were weighed at randomization, on Day 1 (prior to the first dose), and on Day 14 (prior to euthanasia).

Blood samples were collected from all animals prior to euthanasia on Day 14. Animals were anesthetized with an intravenous injection of a ketamine/xylazine cocktail (77 mg/mL ketamine, 23 mg/mL xylazine) at 0.1 mL/kg, and 7 mL of blood was collected via cardiac puncture. The time of blood collection was recorded. Each sample was collected 1 hour 15 minutes after the final dose. Blood was collected into a lavender top tube, agitated for 10 seconds to facilitate adequate mixture of blood and ethylenediaminetetraacetic acid, and then placed on ice until stored refrigerated. Samples were then shipped for pharmacokinetic analyses. Following blood collection, animals were euthanized with an intravenous injection of commercial euthanasia solution per an SOP.

Two animals per study group were randomly selected for histopathologic evaluation of ocular tissues. Tissues were collected for histopathological evaluation as follows: prior to enucleation, both eyes were flushed with 3-5 mL of Balanced Salt Solution (BSS). Both globes were then enucleated. The surrounding tissues, including lacrimal glands and eyelids, were collected as a single complex and placed in 10% neutral buffered formalin. The globes were stored in Davidson's solution for approximately 24 hours and then transferred to 70% ethanol. The time that globes were placed into Davidson's solution and in ethanol were recorded. The globes and the lacrimal glands/eyelids were submitted for histopathological evaluation.

The remaining two animals per study group were used for pharmacokinetics analysis of ocular tissues. Tissues for pharmacokinetics analysis were collected as follows: prior to enucleation, both eyes were flushed with 3-5 mL of BSS. The aqueous humor was collected from each eye, and the volume of aqueous humor was measured. Both globes and surrounding tissues, including lacrimal glands and eyelids, were collected. The lacrimal glands and eyelids were weighed separately. The conjunctiva was collected from each globe and weighed. All collected tissues were snap-frozen in liquid nitrogen. After freezing, the following tissues were collected from each globe: cornea, iris/cilliary body complex, vitreous humor, retina/choroid complex, and sclera. Tissues were collected according to an SOP. Tissues dissected from globes were weighed, labeled, and stored frozen (−70° C.). All ocular tissues were shipped on dry ice for analysis of MPA concentrations.

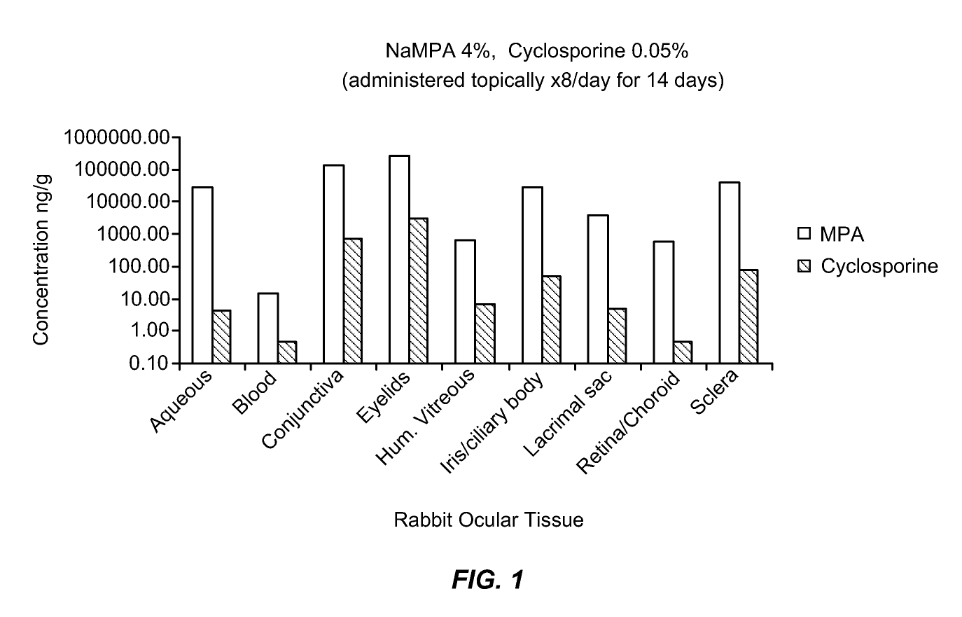

Following topical administration (8 times per day) over 14 days of 4% NaMPA or 0.05% cyclosporine, MPA and cyclosporine levels were measured in different ocular tissues as shown in FIG. 1. Note that the scale is Logarithmic. High Drug concentrations were present in anterior or “front of the eye” structures (e.g., aqueous, conjunctiva, eyelids). Moreover, significant levels of MPA were also found in more posterior or “back of the eye” tissues (e.g., vitreous, retina/choroid). These high levels of MPA penetration into the anterior and posterior eye tissues following topical administration with NaMPA formulations are unexpected as MPA is hydrophilic and lipophobic, and therefore it is not expected to penetrate the cornea. These tissue levels of MPA, either anterior or posterior, are expected to have pharmacologic activity against ocular inflammatory diseases.

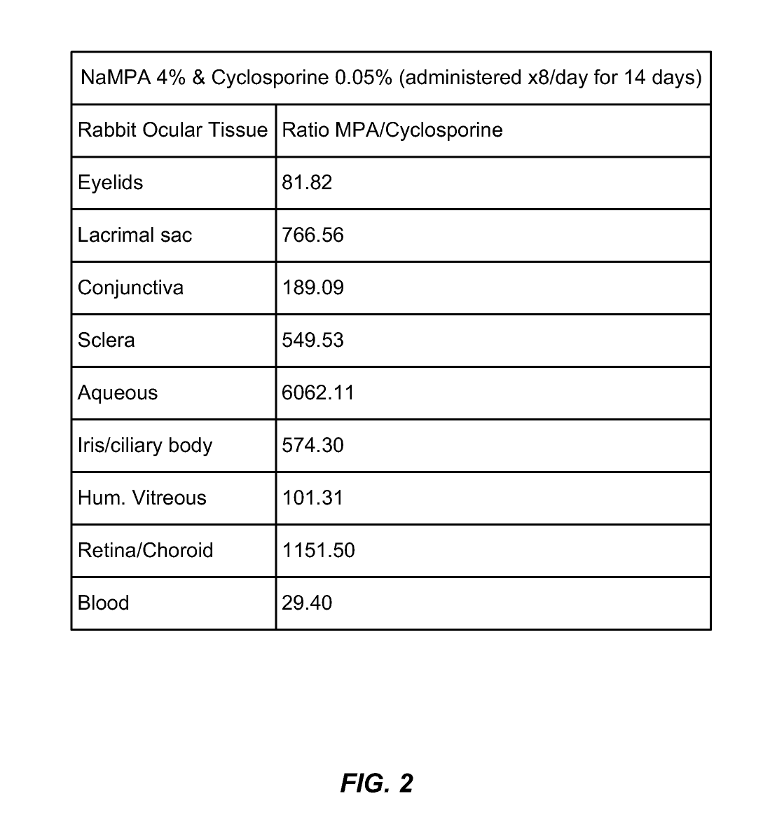

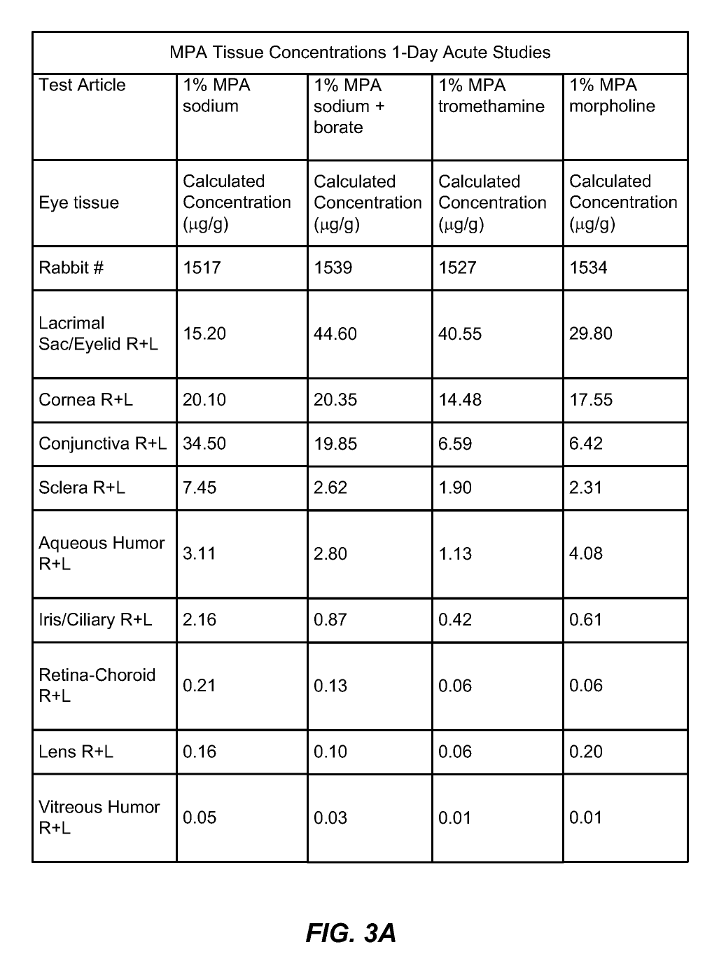

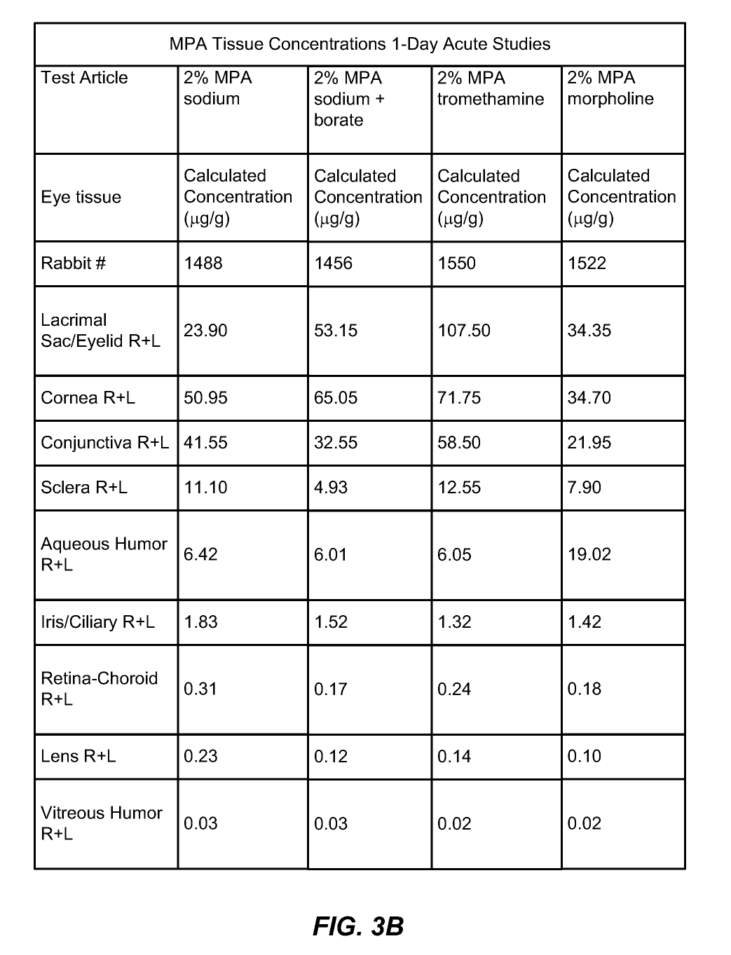

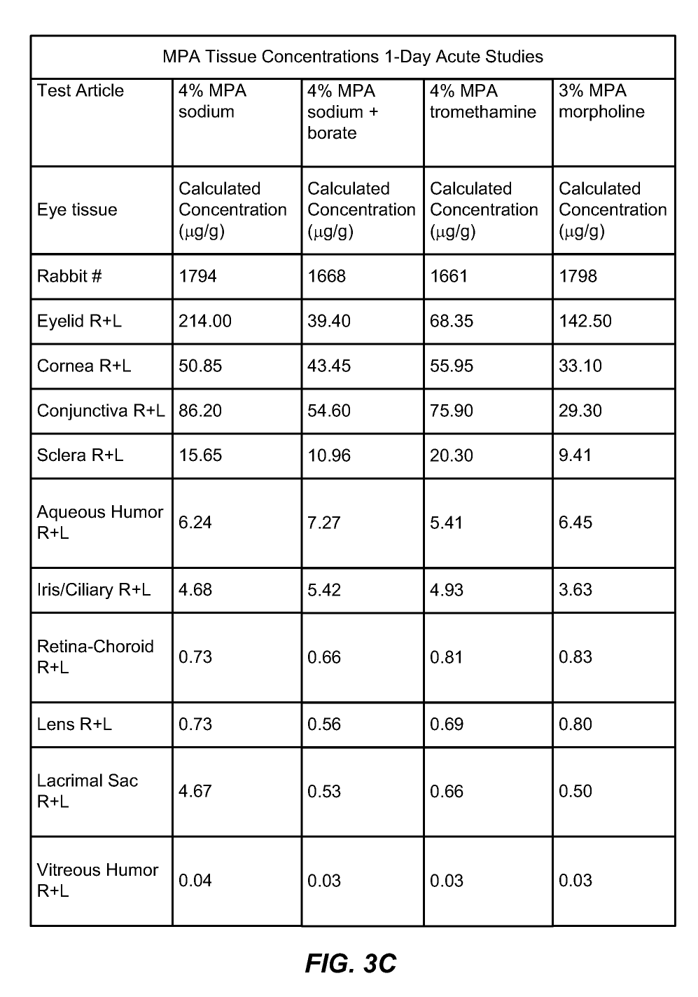

To compare relative ocular tissue penetration between NaMPA formulations and cyclosporine, and to take into account the difference in concentration of active drug (i.e., 4% NaMPA vs. 0.05% cyclosporine), the ratio of tissue concentrations achieved are presented in FIG. 2. In general, these data indicate that ocular penetration following topical administration is greater with the NaMPA formulations than with cyclosporine in many ocular tissues (lacrimal sac, sclera, aqueous, iris/ciliary body and retina/choroid). In the 1-day acute studies, tissue penetration of MPA was observed to be dependent on the concentration of NaMPA in the ophthalmic formulation (1%, 2%, 4% NaMPA), as shown in FIGS. 3A, 3B and 3C.

### Example 5

### Studies on Scopolamine Induced Dry Eye in C57BL/6 Mice

This dry eye model was based on that described in De Paiva et al., 2006, Investigative Opthalmology & Visual Science 47(7):2847-2856, incorporated herein by reference. The experimental design is summarized in Table 8. The study consisted of seven groups of 10 female C57BL/6 mice each. Starting on Day 0, mice of Groups 1-6 were treated four times-daily (QID) with topical bilateral ocular (OU) administration of control or test articles at intervals of at least two hours. Specifically, Groups 1-6 were dosed with (respectively) Vehicle; NaMPA at 0.5, 1.0, or 2.0%; dexamethasone (0.1%); or Restasis® (0.05% cyclosporine). Mice in Group 7 served as untreated controls. On Day 3, scopolamine patches were placed on the tails of the mice and replaced every other day (Q2D) thereafter. From Days 3-12, mice were maintained in an environment with high-volume air draft (see Environmental stress section, below). Necropsies were performed on Day 13.

TABLE 8
Experimental Design

| | | Days 0-12 | | |
|---|---|---|---|---|
| | | Treatment | | |
| | Animal | (QID, OU, 5 μL/ | Days 0-12 | Day 13 |
| Group | Nos. | eye) | In-life Procedures | Terminal Procedures |
| 1 | 151-160 | Vehicle | Clinical observations: QD | Body weights |
| 2 | 251-260 | 0.5% NaMPA | Ophthalmology: pre-dose | Collection, fixation, and |
| 3 | 351-360 | 1.0% NaMPA | and QD from Day 6 | histopathology of eyes, eye |
| 4 | 451-460 | 2.0% NaMPA | Body weights: pre-dose and | lids, conjunctiva, and |
| 5 | 551-560 | Dexamethasone | then 2x per week | lacrimal glands (OU) |
| | | (0.1%) | | |
| 6 | 651-660 | Restasis ® (0.05% | | |
| | | cyclosporine) | | |
| 7 | 751-760 | No treatment | | |

NaMPA Ophthalmic Formulations. Ophthalmic formulations containing 0.5, 1 and 2% NaMPA (w/v) were used (prepared according to Table 1). All NaMPA formulations were stored refrigerated at 2-8° C., protected from light.

Negative Control. The negative control solution was the same vehicle used to formulate the NaMPA solutions, but lacking NaMPA. This solution is referred to as “Vehicle”. Vehicle was stored refrigerated at 2-8° C., protected from light.

Positive Controls. The first positive control was an ophthalmic suspension of 0.1% dexamethasone (dexamethasone ophthalmic suspension USP, Henry Schein (Melville, N.Y.) Catalog 1033542). Dexamethasone was stored at controlled room temperature (20-25° C.) per manufacturer's instructions.

The second positive control was an ophthalmic emulsion of 0.05% cyclosporine (Restasis®; Allergan, Inc., Irvine, Calif.). Restasis® was stored at controlled room temperature (20-25° C.) per manufacturer's instructions.

Animals. A total of 78 female C57BL/6 mice (Mus musculus), 16-25 g each upon arrival, were purchased from Charles River Laboratories (Hollister, Calif.) for use in the study.

Dose Administration. On each day of dosing (Days 0-12), NaMPA or control formulations were administered topically to both eyes of each animal four times per day, with a minimum of two hours between treatments (5 μL/eye/dose, OU, QID). Specifically, Groups 1-6 were dosed with (respectively) Vehicle; NaMPA at 0.5, 1.0, or 2.0%; dexamethasone (0.1%); or Restasis®. Mice of Group 7 served as untreated controls.

Induction of Dry Eye. Bilateral, short-term, reversible dry eye was induced in mice by skin patch administration of scopolamine, combined with maintenance of mice in an environment with high-volume air draft from Days 3 through 12.

Depilation. Prior to the first scopolamine dose (Day 3), fur around the base of the tail was removed with depilatory.

Scopolamine Dosing. Scopolamine patches were obtained as transdermal scopolamine patches (Henry Schein Catalog 2482592). Full-size patches contain 1.5 mg scopolamine each; therefore, for each dose, individual patches were cut in two, and half a patch (˜0.75 mg) was applied to each animal.

For each day of administration, mice were briefly restrained. Starting on Day 3 (i.e., the fourth day of ocular treatment with NaMPA or control formulations), and repeating every other day thereafter (Q2D), half a transdermal scopolamine patch was placed on the tail. Following each application, the patch was wrapped with Vetwrap® to prevent removal by the animals, and left in place for up to 48 hr. Following patch application, animals were monitored for any adverse reactions, including the condition of the tail and Vetwrap®.

Environmental Stress. Following the start of scopolamine dosing, mice were placed in an environment with high-volume air draft (i.e., air draft at a setting of one with the blower on in a laminar flow hood) for up to eight hours/day on Days 3 through 12.

Clinical Observations. Clinical observations, including overt signs of toxic or pharmacologic effect(s), were conducted at least once daily for each animal during the acclimation and treatment periods. All abnormal clinical signs were recorded.

Body Weights. All animals were weighed prior to the first test/control article dose, twice weekly thereafter, and at necropsy.

Opthalmology. Prior to inclusion on study, both eyes of each animal were examined. Pre-dose assessments included gross ocular observations; slit lamp corneal examinations; and phenol red thread tear tests. Mice with signs of ocular irritation or abnormality were not entered onto the study. Following the start of test/control article dosing, corneal examinations were performed once daily from Days 6 to 12 (QD, OU), except on Day 8; tear tests were performed daily from Day 1 (QD, OU).

Gross Ocular Observations. Both eyes of each animal were evaluated for signs of irritation or discomfort. If any abnormalities were observed, such signs were recorded on a standardized data collection sheet.

Slit Lamp Corneal Examinations. Both eyes of each animal were evaluated via a slit lamp ophthalmic examination using topically-applied fluorescein. Fluorescein dye was used to examine the surface of the cornea and scored.

Necropsy. Animals were euthanized immediately prior to necropsy. At necropsy, both eyes from each animal, including eyes, eye lids, lacrimal glands, and conjunctiva, were excised. These tissues were fixed in 10% Neutral Buffered Formalin.

Pathology. Following fixation, microscopic evaluation was performed on both eyes from each animal. At least two section levels were examined histopathologically for each eye. Tissues were dehydrated, embedded in paraffin, serial-sectioned (at 3- to 5-μm thickness), and stained with hematoxylin and eosin. A board-certified veterinary pathologist evaluated slides via light microscopy. Detailed and complete histopathologic assessment of all parts of the eye was performed, with special attention to the cornea, epithelia (including goblet cells) of the conjunctiva and cornea, and lacrimal glands, to identify histopathology consistent with dry eye, keratitis, or other changes in the cornea, if present.

Eyes were serially sectioned and examined. The representative corneas were scored based upon a 0-4 scale, with 0 being normal, 1 being minimal, 2 being mild, 3 being moderate, and 4 being severe. For each cornea, scores were assigned for each of the following parameters: (a) corneal edema (presence of edema in the corneal stroma); (b) epidermal thickness (the number of epidermal cells in the epidermal layer counted from basal cells to superficial cells); and (c) epidermal cell edema (presence of intra-epithelial cell edema). Scoring for epidermal cell thickness represents the mean number of cell layers present for a given animal cohort, which in this analysis ranged from 2-6. Therefore, maximal scoring for this parameter can exceed 4.

Statistical Analysis. Calculations and descriptive statistics (means, standard deviations) were performed using Excel# (Office 2007; Microsoft, Redmond, Wash.). Where appropriate, inferential statistical analysis was performed using either Excel# or Prism# (Version 5.01; GraphPad Software, Inc., San Diego, Calif.). P-values of 0.05 or less (P<0.05) were considered statistically significant.

Continuous Normal Data. For more than two groups, Bartlett's Test for Equal Variances was used to determine homogeneity of the data from multiple dosing groups. Where variance was homogeneous, One Way Analysis of Variance (ANOVA) was used, followed by Tukey's Multiple Comparisons post-hoc test if the ANOVA was significant. For non-homogeneous variance, the Kruskal-Wallis (non-parametric) test was used, followed by Dunn's post-test if the Kruskal-Wallis test was significant.

Categorical Data. Histopathology lesion data was analyzed by the Study Pathologist. Where appropriate, histopathology severity scores were analyzed statistically using non-parametric tests.

Results. Ophthalmic NaMPA formulations, even at the highest concentration used in this study, did not produce any notable ocular irritation or adverse clinical signs, when administered topically four times daily for 13 days. Therefore, these ophthalmic NaMPA formulations were well tolerated in mice prior to dry eye induction (i.e., under normal conditions) as well as when clinical signs of dry eye were present.

In the histopathology analysis (see Table 9), there was a reduction in corneal injury in the 2% NaMPA group. This effect reached statistical significance (p<0.05) for corneal edema vs. Vehicle control, and for epidermal cell thickness versus the no treatment group. For epidermal cell edema, the mean scores in the 2% NaMPA group fell to approx. 63% of those in the Vehicle control group, although this reduction did not reach statistical significance. Overall, the reduction in histopathological findings in the 2% NaMPA group was considered biologically significant.

The positive control, dexamethasone, also reduced injury to the cornea. This effect reached statistical significance (p<0.05) for corneal edema and epidermal cell thickness versus Vehicle control, and for corneal edema, epidermal cell thickness and epidermal cell edema versus the no treatment group. However, with regard to adverse effects, dexamethasone treated animals experienced approx. 20% weight loss relative to other groups by the end of the study (Day 13). A second positive control, Restasis®, did not have a protective effect in this study by any of the measured parameters.

The 2% NaMPA ophthalmic formulation was demonstrated to be efficacious in the scopolamine-induced model of dry eye. This efficacy was based on histopathology analysis, where statistically significant reductions in corneal edema and epidermal cell thickness versus negative controls was observed. There were also trends toward reductions in histopathological scoring for the 1% NaMPA group, suggesting a dose-responsive effect. Therefore, this data indicates that ophthalmic NaMPA formulations are effective in reducing ocular histopathology in a murine model of dry eye, and are well tolerated even when signs of dry eye are present. This data demonstrates that ophthalmic NaMPA formulations have potential for the treatment of dry eye in humans.

TABLE 9
Histopathology Scoring of the Cornea (summary)

| | | Epidermal Cell | Epidermal Cell |
|---|---|---|---|
| Group | Corneal Edema | Thickness | Edema |
| 1 | 3.5 ± 0.7 | 4.1 ± 1.1 | 1.6 ± 0.5 |
| Vehicle | | | |
| 2 | 2.5 ± 1.0 | 3.4 ± 0.8 | 1.1 ± 0.3 |
| NaMPA (0.5%) | | | |
| 3 | 2.4 ± 0.7 | 3.2 ± 0.4 | 1.1 ± 0.9 |
| NaMPA (1.0%) | | | |
| 4 | 2.2* ± 0.4 | 3.0^{#} ± 0.5 | 1.0 ± 0.7 |
| NaMPA (2.0%) | | | |
| 5 | 1.6*^{#} ± 0.5 | 2.8*^{#} ± 0.6 | 0.8^{#} ± 0.4 |
| dexamethasone | | | |
| 6 | 3.1 ± 0.6 | 4.0 ± 0.0 | 2.3 ± 0.7 |
| Restasis ® | | | |
| 7 | 2.8 ± 0.6 | 4.4 ± 1.3 | 1.9 ± 0.6 |
| No treatment | | | |

*Statistically significant (P < 0.05) compared to Group 1.
^{#}Statistically significant (P < 0.05) compared to Group 7.

### Example 6

### Studies Using Con-A Induced Dry Eye

To assess the activity of the NaMPA containing ocular solutions, dry eye was induced in rabbits by bilateral injection of concanavalin A (Con A) into the lacrimal glands. Treatment with the ocular solutions is as described below. Ocular examination, clinical tests and histopathology were conducted to assess the effects of treatment on dry eye.

The experimental design is summarized in Table 10. The study consisted of seven groups of 6 male NZW rabbits each. Prior to group assignment, animals were examined by veterinary and opthalmological examinations; rabbits with any signs of abnormality were excluded. Animals of Groups 1-5 were dosed with (respectively) Vehicle; NaMPA at 0.5, 1.0, or 2.0% (w/v); or 0.05% cyclosporine (Restasis®). These animals were dosed by bilateral ocular topical application (OU eye drops) four times daily (QID; at least 2 hr between each dose) on Days 0-14, except for Day 8, when only the last two doses of the respective NaMPA/control solutions were administered. Animals of Group 6 were dosed with OU QID 0.1% dexamethasone eye drops on Days 9-14. Group 7 rabbits received no eye drops and served as untreated controls. On Day 8, all animals (Groups 1-7) were dosed bilaterally with 300 μg (in 50 μL) per eye of Con A, administered by injection into the accessory lacrimal glands (see Con A injection procedure, below).

Clinical observations were recorded once daily prior to the start of topical ocular dosing (before Day 0); immediately before and after the first and fourth daily dose of NaMPA or control solutions (Days 0-16); and prior to sacrifice (Day 17). Any signs of abnormality, especially ocular inflammation or irritation, were documented. The Tear Break-up Test (TBUT) was conducted as follows: for three consecutive days (Day-2 through Day 0) prior to start of dosing with NaMPA or control solutions; three times during the first week (Day 1 through Day 7) preceding Con A injection; and once daily in the interval (Day 9 through 17) following Con A injection. No TBUT was conducted on the day of Con A injection (Day 8). Body weights were recorded prior to the start of topical ocular dosing; twice weekly during the treatment period (Days 0-16); and prior to sacrifice (Day 17). On Day 17, rabbits were euthanized.

TABLE 10
Experimental Design

| | | Days 0-16 | | | |
|---|---|---|---|---|---|
| | Animal | Treatment | Day 8 | | Day 17 |
| | No. | (QID*, OU, | Dry-eye | Through Day 16 | Terminal |
| Group | (males) | 25 μL/eye) | Induction | In-life Procedures | Procedures |
| 1 | 101-106 | Vehicle | Injection of | Body weight: | Body weight |
| 2 | 201-206 | 0.5% NaMPA | Con A (300 μg | pre-dose | |
| 3 | 301-306 | 1% NaMPA | in 50 μL per | 2x/wk on Days 0-16 | |
| 4 | 401-406 | 2% NaMPA | eye, OU) | Clinical obs: | |
| 5 | 501-506 | Restasis ® | | QD pre-dose | |
| | | (0.05% | | QID on Days 0-16 | |
| | | cyclosporine) | | TBUT: | |
| 6 | 601-606 | 0.1% | | QD pre-dose | |
| | | dexamethasone | | 3x/wk on Days 0-7 | |
| | | (Days 9-14 | | QD on Days 9-17 | |
| | | only) | | | |
| 7 | 701-706 | No treatment | | | |

*Groups 1-5: only 2 doses on Day 8 (after Con A injection). Group 6: dosing started on Day 9 (after Con A injection).

Concanavalin A (Con A, Sigma-Aldrich, St. Louis, Mo., Catalog C5275) was used in this study. Con A was stored at −20° C.

Ophthalmic formulations containing 0.5%, 1% and 2% NaMPA (w/v) (prepared according to Table 1) were used in this study. All NaMPA solutions were stored refrigerated at 2-8° C., protected from light.

Negative Controls. The first negative control was the same vehicle used to formulate the NaMPA solutions, but lacking NaMPA. This control solution was referred to as “Vehicle” (or in some instances, “V-NE”). Vehicle was stored refrigerated at 2-8° C., protected from light.

The second negative control was phosphate-buffered saline (PBS, MP Biomedicals, Solon, Ohio, Catalog 1860454; Dulbecco's Formula, without Magnesium and Calcium). PBS was stored refrigerated at 2-8° C.

Positive Controls. The first positive control was an ophthalmic suspension of 0.1% dexamethasone (USP, Henry Schein, Melville, N.Y., Catalog 1033542). Dexamethasone was stored at controlled room temperature (20-25° C.) per manufacturer's instructions. The second positive control was an ophthalmic emulsion of 0.05% cyclosporine (Restasis®, Allergan, Inc., Irvine, Calif.). Restasis# was stored at controlled room temperature (20-25° C.) per manufacturer's instructions.

Dose Preparation. Prior to use in formulation and dosing, the pH of the sterile PBS was adjusted to ˜6.8 and filter-sterilized through a 2-μm filter. The pH-adjusted PBS was allowed to warm to room temperature (for up to 1 hr) on the bench-top prior to use. Con A was formulated and diluted in sterile PBS (pH 6.8) to a final concentration of 6 mg/mL. Con A dosing solutions were inspected for physical state (hazy, clear, etc.) prior to use, but were not filtered. Immediately prior to each dose administration, Con A solutions were gently inverted to ensure that the solution/suspension was well mixed.

Animals. A total of 46 male NZW rabbits (Oryctolagus cuniculus), 2.4-2.6 kg each upon arrival, were purchased from Myrtle's Rabbitry (Thompsons Station, Tenn.) for use in the study.

Final Selection and Group Assignment. Following acclimation and prior to the start of dose administration, body weights were recorded, and animals were subjected to pre-treatment veterinary and opthalmological evaluations, including gross ocular observation, slit lamp corneal examination, and TBUT. Following evaluation, animals were released for use in the study. Forty-two of the animals that were in the desired weight range were selected and arbitrarily assigned to seven study groups of six rabbits each. Animals were selected based on body weight and normal clinical and ocular presentation. Animals were also selected to be included in the study based on baseline TBUT values that were “within the normal range” and “less variable” during the three baseline testing periods.

Dose Administration. Animals of Groups 1-5 were dosed with (respectively) Vehicle; NaMPA at 0.5, 1.0, or 2.0% (w/v); or 0.05% cyclosporine (Restasis®). These animals were dosed by bilateral ocular topical application (OU eye drops) four times daily (QID; at least 2 hr between each dose) on Days 0-16, except for Day 8, when only the last two doses of the respective NaMPA/control solutions were administered. Animals of Group 6 were dosed with OU QID 0.1% dexamethasone eye drops on Days 9-16. Rabbits of Group 7 received no eye drops and served as untreated controls.

Pre-treatment Opthalmological Examinations. The animals were screened prior to the first dose administration to exclude animals with pre-existing ocular conditions. All animals were found to be within opthalmologically normal limits and released for inclusion in the study.

Induction of Dry Eye. Bilateral, short-term, reversible dry eye was induced in NZW rabbits by injecting (on Day 8) Con A into the lacrimal glands.

Con A injection Procedure. Rabbits were briefly anesthetized with ketamine:xylazine (45:5 mg/kg). Con A was injected into both eyes of each animal at a per-eye dose of 300 μg (50 μL at 6 mg/mL). Con A was injected through the fomix into the lacrimal gland bilaterally using a 1 ml tuberculin syringe with a 30-gauge, 1½-inch needle. The needle was introduced 1 cm from the nasal canthus along the slightly retracted lower eyelid, in the suborbital space, to a depth of about 15 mm. The injection was made downward inside the orbit and then around behind the eye. The method of administration remained consistent throughout the study.

Clinical Observations. Clinical observations, including overt signs of toxic or pharmacologic effect(s), were recorded once daily prior to the start of topical ocular dosing; immediately before and after the first and fourth daily dose of test or control article (Days 0-14); and prior to sacrifice (Day 15). Any signs of abnormality, especially ocular inflammation or irritation, were documented.

Body Weights. All animals were weighed prior to the first topical ocular dose administration, twice weekly thereafter, and at sacrifice. Dexamethasone-dosed rabbits were weighed more frequently, as necessary, to assess possible weight loss.

Opthalmology. Prior to inclusion on study, both eyes of each animal were examined to assure that the eyes were within normal limits. Pre-dose assessments (on Day-1) included gross ocular observations, slit lamp corneal examinations and TBUT. Rabbits with abnormal anterior segments were not included in the study.

For Days 0-16, gross ocular observations were performed as part of the clinical observations (i.e., up to four times daily) For Days 0-17, TBUT tests were conducted as follows: for three consecutive days (Day-2 through Day 0) prior to start of dosing with NaMPA or control article; three times during the week preceding Con A injection (Day 1 through Day 7); and once daily following Con A injection (Day 9 through 17). No TBUT testing was conducted on the day of Con A injection (Day 8). TBUT testing was conducted prior to topical dosing.

Gross Ocular Observations. Both eyes of each animal were evaluated for signs of irritation or discomfort. If any abnormalities were observed, such signs were recorded on a standardized data collection sheet.

Tear Break-up Test (TBUT). TBUT values were determined after instilling 5 μL of 2% sterile sodium fluorescein evenly across the surface of the eye. Lids then were manually blinked once (to evenly distribute the fluorescein in the tear film) and the eye lids were held open (to prevent additional blinking). Each eye was illuminated under cobalt light illumination, and a slit lamp was used to measure the time (in seconds) required for one or more dark holes or streaks to appear in the fluorescein-tear film covering the eye.

Statistical Analysis. All statistical analyses were done as described under Studies on Scopolamine Induced Dry Eye in C57BL/6 Mice.

Results: In this example, a Con-A-induced dry eye model was performed in rabbits (agelhout et al., 2005, Journal of Ocular Pharmacology and Therapeutics 21(2):139-148). Clinical measurement of dry eye was done using the tear break up time (TBUT) assay. Topical treatments with Vehicle, NaMPA and Restasis® were administered from Day 0 to Day 17, while dexamethasone was given from Day 9 to Day 17. With the exception of a single day (Day 8), when given 2 times daily, treatments were given four times daily. Dry eye induction was initiated on Day 8 by bilateral lacrimal gland injection of concanavalin A (Con A).

Ophthalmic NaMPA formulations, even at the highest concentration used in this study, did not produce any notable ocular irritation or adverse clinical signs, when administered topically for 17 days.

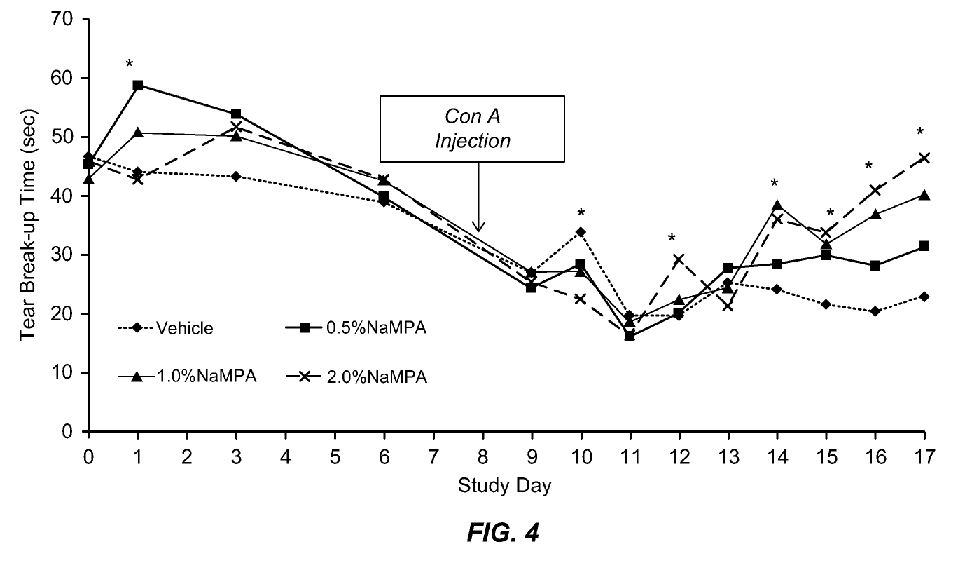

Following Con A injection, dry eye was induced as indicated by a reduction in TBUT values in the Vehicle group beginning Day 11 and continuing to the end of study, Day 17. On Days 14 through 17, TBUT values were significantly increased (P<0.05 to P<0.001) in the 2% NaMPA and 1% NaMPA groups vs. the Vehicle group. On Day 15, the TBUT value was significantly increased (P<0.05) in the 0.5% NaMPA group vs. the Vehicle group (Table 11 and FIG. 4).

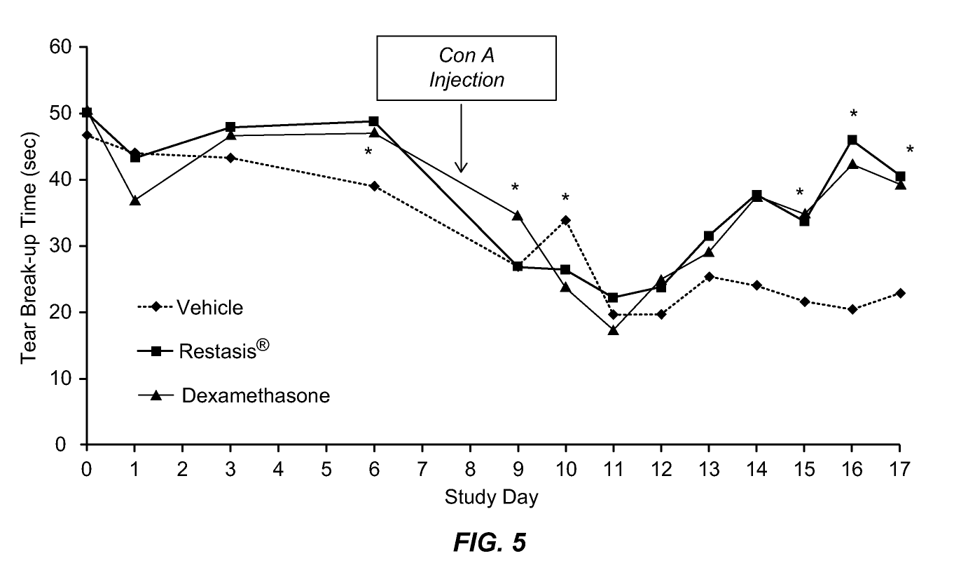

Similarly, on Days 14 through 17, TBUT values were significantly increased (P<0.01 to P<0.001) in the Restasis® and dexamethasone groups vs. the Vehicle group (Table 12 and FIG. 5). This data indicates that topical administration of all three concentrations of the NaMPA ophthalmic solutions resulted in significant improvement in TBUT values during the induction of dry eye. The increase in TBUT value noted for Days 14-17 was dose-responsive for the NaMPA groups, returning to or approaching the baseline values observed prior to Con A injection by Day 17. In addition, the improvement in TBUT values observed over Days 14-17 for the 2% and 1% NaMPA groups was similar to that seen for the two positive controls, dexamethasone and Restasis®.

These results indicate that the ophthalmic NaMPA formulations are highly active in correcting the TBUT parameter of dry eye in this model, and are equivalent in efficacy to the prescription drug approved for human dry eye treatment, Restasis®, as well as the ophthalmic steroid, dexamethasone. Measurement of TBUT is an accepted clinical criteria for diagnosis of dry eye in humans (Report of the International Dry Eye Workshop (DEWS). The Ocular Surface. April 2007, Vol. 5, No. 2 pg. 65-152). TBUT is a standard measurement of tear film stability, which in turn is related to the composition of the tear film, including mucins and lipids. Premature break-up of the tear film, which is reflected by a decreased TBUT value, is a feature of any form of dry eye (Kallarackal et al. Eye (2002) 16: 594-600). Abnormalities in the quality of tear film can result in symptomatic dry eye even when the quantity of aqueous tear production is normal (Lemp et al., 1971, Trans Am Acad Opthalmol Otolaryngol 75:1223-1227). Hence, TBUT alterations, even in isolation, are of clinical significance in dry eye. Improvement of TBUT values by NaMPA treatment is therefore predictive for efficacy in human dry eye. In addition to being effective in this dry eye model, the ophthalmic NaMPA formulations were well tolerated by animals under normal conditions and during the period when signs of dry eye were present (i.e., TBUT reduction). This is an important consideration for ophthalmic treatments, where topical drug tolerability can be reduced in ocular inflammatory disease, as is the case for human dry eye. Collectively, this data demonstrates the potential for ophthalmic NaMPA formulations in the treatment of dry eye in humans.

TABLE 11
TBUT Statistical Results for NaMPA Groups

| Grp 1-4 | D0 | D1 | D3 | D6 | D9 | D10 | D11 | D12 | D13 | D14 | D15 | D16 | D17 | Post-hoc comparison |
|---|---|---|---|---|---|---|---|---|---|---|---|---|---|---|
| Overall P | NS | 0.0335 | NS | NS | NS | 0.0012 | NS | 0.0212 | NS | 0.0016 | 0.0009 | <0.0001 | <0.0001 | |
| Tukey 1 vs 2 | | NS | | | | NS | | NS | | NS | <0.05 | NS | NS | Vehicle vs. 0.5% |
| | | | | | | | | | | | | | | NaMPA |
| Tukey 1 vs 3 | | NS | | | | <0.05 | | <0.05 | | <0.01 | <0.01 | <0.01 | <0.001 | Vehicle vs. 1% |
| | | | | | | | | | | | | | | NaMPA |
| Tukey 1 vs 4 | | NS | | | | <0.001 | | NS | | <0.05 | <0.001 | <0.001 | <0.001 | Vehicle vs. 2% |
| | | | | | | | | | | | | | | NaMPA |
| Tukey 2 vs 3 | | NS | | | | NS | | NS | | NS | NS | NS | NS | |
| Tukey 2 vs 4 | | <0.05 | | | | NS | | <0.05 | | <0.05 | NS | <0.05 | <0.01 | 0.5% vs. 2% NaMPA |
| Tukey 3 vs 4 | | NS | | | | NS | | NS | | NS | NS | NS | NS | |

Data are the average of TBUT values for both eyes for each animal per group. Data was analyzed via Prism 5 software. Statistical comparisons between groups was done using one-way ANOVA, and post-hoc Tukey if applicable.

TABLE 12
TBUT Statistical Results for Restasis ® and Dexamethasone Groups

| Grp 1, 5, 6 | D0 | D1 | D3 | D6 | D9 | D10 | D11 | D12 | D13 | D14 | D15 | D16 | D17 | Post-hoc comparison |
|---|---|---|---|---|---|---|---|---|---|---|---|---|---|---|
| Overall P | NS | NS | NS | 0.0009 | 0.0064 | 0.0016 | NS | NS | NS | 0.0003 | 0.0009 | <0.0001 | <0.0001 | |
| Tukey 1 vs 5 | | | | <0.01 | NS | <0.05 | | | | <0.001 | <0.01 | <0.001 | <0.001 | Vehicle vs. Restasis ® |
| Tukey 1 vs 6 | | | | <0.01 | <0.05 | <0.01 | | | | <0.001 | <0.01 | <0.001 | <0.001 | Vehicle vs. Dexamethasone |
| Tukey 5 vs 6 | | | | NS | <0.05 | NS | | | | NS | NS | NS | NS | Restasis ® vs. Dexamethasone |

Data are the average of TBUT values for both eyes for each animal per group. Data was analyzed via Prism 5 software. Statistical comparisons between groups was done using one-way ANOVA, and post-hoc Tukey if applicable.

### Example 7

### Studies Using Bovine Ocular Melanin Induced Uveitis in Lewis Rats

This study assessed the activity of the NaMPA containing ocular solutions for treatment of uveitis. Uveitis was induced in Lewis rats by injecting the animals with bovine ocular melanin. Treatments were given as described below. Ocular examination and histopathology was conducted to assess the effects of treatment on uveitis.

The experimental design is summarized in Table 13. The study consisted of eight groups of male Lewis rats, i.e., seven groups of 16 rats each (Groups 1 to 7) and one group of eight rats (Group 8). On Day 0, uveitis was induced in each animal by intradermal (ID) injection with an emulsion of bovine ocular melanin, Complete Freund's Adjuvant (CFA), and pertussis toxin. Starting on the same day, Day 0, treatment with NaMPA or control solutions was initiated, as follows. Rats of Groups 1-5 and 8 received four times-daily (QID) topical bilateral ocular administrations, at intervals of at least two hours, of (respectively) Vehicle; NaMPA at 0.5, 1.0, or 2.0%; dexamethasone (0.1%); or Restasis® (0.05% cyclosporine A (CsA)). Rats of Group 6 received once-daily intramuscular (IM) injections of CsA (Sandimmune®). Rats of Groups 5, 6 and 8 served as positive controls (dexamethasone and cyclosporine A) and rats of Group 7 served as untreated controls.

TABLE 13
Experimental Design

| | | Days 0 to 18 or 30/32 | | | | Day 18 or 30/32 |
|---|---|---|---|---|---|---|
| | Animal No. | | Dose and | | | Terminal Procedures |
| Group | (males) | Treatment | Route | Frequency | In-life Procedures | (8 rats/group/day) |
| 1 | 101-116 | Vehicle | 10 μL/eye | QID | Clinical | Body weights |
| 2 | 201-216 | 0.5% | both eyes | | observations: | |
| | | NaMPA | | | prior to start of | |
| 3 | 301-316 | 1.0% | | | study, daily from | |
| | | NaMPA | | | Day 0 | |
| 4 | 401-416 | 2.0% | | | Ophthalmology: | |
| | | NaMPA | | | prior to start of | |
| 5 | 501-516 | Dexamethasone | | | study, 3x per | |
| | | (0.1%) | | | week from Day 7 | |
| 6 | 601-616 | CsA | 15 mg/kg, | QD | Body weights: | |
| | | (15 mg/kg) | IM | | prior to start of | |
| 7 | 701-716 | No treatment | — | — | study, Day 0, then | |
| | | | | | 2x per week | |
| 8 | 801-808 | Restasis ® | 10 μL/eye | QID | | |
| | | (0.05% | both eyes | | | |
| | | CsA) | | | | |

NaMPA Formulations. Ophthalmic formulations of 0.5%, 1% and 2% NaMPA (w/v) (prepared according to Table 1) were used in this study. NaMPA solutions were stored refrigerated at 2-8° C. and protected from light.

Negative Control. The negative control was the same vehicle used to formulate the NaMPA solutions but lacking NaMPA. This control is referred to as “Vehicle” (or “V-NE” in some instances). Vehicle was stored refrigerated at 2-8° C. and protected from light.

Positive Controls. The first positive control was an ophthalmic suspension of 0.1% dexamethasone (USP, Henry Schein (Melville, N.Y.) Catalog 1033542). Dexamethasone was stored at controlled room temperature (20-25° C.) per manufacturer's instructions.

The second positive control was an injectable emulsion of 50 mg/mL cyclosporine A (Cyclosporine Injection (Sandimmune®), USP, Henry Schein (Melville, N.Y.) Catalog 1100667). Injectable cyclosporine was stored at controlled room temperature (20-25° C.) per manufacturer's instructions.

The third positive control was an ophthalmic emulsion of 0.05% cyclosporine (Restasis® (, Allergan, Inc., Irvine, Calif.). Restasis® was stored at controlled room temperature (20-25° C.) per manufacturer's instructions.

Animals. A total of 124 male Lewis rats (Rattus norvegicus; strain LEW/SsNHsd), 160-180 g each upon arrival, were purchased from Harlan Laboratories (Indianapolis, Ind.) for use in the study.

Topical Ocular Administration. Groups 1-5 and 8 were dosed with (respectively) Vehicle; NaMPA at 0.5, 1.0, or 2.0%; dexamethasone (0.1%); or Restasis® (0.05% CsA). On each day of dosing, NaMPA or control solutions were administered topically to both eyes of each animal four times per day, with a minimum of two hours between treatments (10 μL/eye/dose, OU, QID). On Days 15/16 the dosing regimen for Group 5 was reduced to BID for the remainder of the study. This was in response to the significant weight loss in this group.

Parenteral Administration. Rats of Group 6 received once-daily intramuscular injections of CsA at 15 mg/kg. Injections were delivered at 0.3 mL/kg, with dose volumes recalculated regularly based on the most recent body weight and administered to left and right hind-limbs on alternating days.

Induction of Uveitis. Bilateral experimental autoimmune anterior uveitis was induced in rats by systemic administration of an emulsion containing a bovine ocular extract (Broekhuyse et al., 1991, Exp Eye Res 52:465-474). Herein, this preparation is also referred to as “melanin-associated antigen” (MAA). MAA was isolated from bovine eyes and diluted in phosphate-buffered saline (PBS) at a concentration of 1.468 mg/mL and stored at −20° C.

Emulsion. On the day of immunization (Day 0), an immunogenic emulsion composed of MAA, CFA, and pertussis toxin was prepared as follows. CFA (containing 4 mg/mL heat-killed Mycobacterium tuberculosis (MTB)) was purchased from Chondrex (Redmond, Wash.) as Catalog 7001. CFA was stored at 4° C. pending use in immunization. Pertussis toxin was purchased from Sigma-Aldrich (St. Louis, Mo.) as Catalog P7208 (pertussis toxin from Bordetella pertussis). Toxin was stored refrigerated at 2-8° C. pending use in immunization. Immediately before use, the contents of each vial was reconstituted in 0.5 mL (i.e., 100 μg/mL) of sterile water. For the preparation of emulsion, MAA, CFA, and toxin were combined at ratios of 7:7:1 (2.34 mL: 2.34 mL: 0.33 mL, respectively) and emulsified by multiple passages through a Hamilton emulsifying needle apparatus for at least 30 min.

Injection. Rats were briefly restrained and injected with the emulsion intradermally (ID) into the tail head (˜150 μL/rat). As formulated above, this injection corresponded to a per-rat dose of 100 μg of MAA, 292 μg MTB, and 1 μg of toxin.

Opthalmology. Prior to inclusion on study, both eyes of each animal were examined to assure that the eyes were within normal limits. Rats with signs of ocular irritation or abnormality were not entered onto the study. From Day 7 of the treatment period, and continuing throughout the in-life, slit lamp examinations were performed three times per week.

Ocular Observations. Both eyes of each animal were evaluated for signs of irritation or discomfort. Observations were recorded using a standardized data collection sheet.

Slit Lamp Ophthalmic Examinations. Both eyes of each animal were evaluated via a slit lamp ophthalmic examination. The anterior segment of each eye was examined and any changes were recorded and scored, with respect to hyperemia and opacity, using standardized data collection sheets.

Statistical Analysis. All statistical analyses were done as described in Studies on Scopolamine Induced Dry Eye in C57BL/6 Mice.

Results.: In this example, a model of experimental melanin-induced uveitis (EMIU) was performed in the Lewis rat (Smith et al., 2008, Ophthalmic Research 40: 136-140). In this model, anterior uveitis is induced by systemic immunization with bovine ocular melanin-associated antigen (MAA).

As determined by ophthalmic examination, the appearance of anterior segment inflammation typically began around Days 14-15, reached maximal scoring values around Days 16-18, plateaued during Days 19-20 and declined thereafter through to Days 28/29, the last days of observation.

Ophthalmic NaMPA formulations, even at the highest concentration used in this study, did not produce any notable ocular irritation or adverse clinical signs when administered topically four times daily for up to 30 days. Therefore, these ophthalmic NaMPA formulations were well tolerated in rats prior to the appearance of clinical signs of uveitis (i.e., under normal conditions), as well as during the period of uveitis, when uniform and brisk inflammation of the anterior segment was present as determined by ophthalmic examinations.

Administration of the positive controls dexamethasone and cyclosporine (intramuscular injection) as well as 2% NaMPA resulted in statistically significant reduction of the ocular scores for both hyperemia and corneal opacity (P<0.05) vs Vehicle control (Tables 14 and 15). For 2% NaMPA, this reduction occurred late in the uveitis observation period (Days 28/29), indicating a slightly more rapid resolution of anterior inflammation. For dexamethasone and parenteral cyclosporine, reductions were observed both early (Days 14/15 and Days 17/18) as well as late (Days 28/29) in the observation period, indicating reductions in peak uveitis scores as well as more rapid resolution of anterior inflammation. However, dexamethasone-treated rats experienced significant drug-related weight loss during study, an observation also noted in mice treated with this ophthalmic steroid (see Studies on Scopolamine Induced Dry Eye in C57BL/6 Mice). In addition, in the Restasis® (topical 0.05% cyclosporine) group (sacrificed after Day 18), there were no significant effects on any of the ophthalmic scoring parameters for anterior segment inflammation.

Based on the statistically significant effect of the ophthalmic 2% NaMPA formulation on the hyperemia and corneal opacity parameters of anterior segment inflammation, combined with a good tolerability profile in rats with anterior uveitis, these results demonstrate that topical NaMPA formulations have potential as therapeutics for human uveitis.

TABLE 14
Hyperemia Scores (pooled)

| | Days (score) | | | | | |
|---|---|---|---|---|---|---|
| Group | −2/−1 | 14/15 | 17/18 | 19/20 | 26/27 | 28/29 |
| 1 | 0 | 3.2 ± | 3.6 ± 0.5 | 3.0 ± 0.0 | 1.8 ± 0.4 | 1.0 ± 0.0 |
| | | 0.5 | | | | |
| 2 | 0 | 3.0 ± | 4.0 ± 0.0 | 3.0 ± 0.0 | 2.0 ± 0.0 | 0.9 ± 0.4 |
| | | 0.4 | | | | |
| 3 | 0 | 2.9 ± | 4.0 ± 0.0 | 3.0 ± 0.0 | 2.0 ± 0.0 | 0.7 ± 1.0 |
| | | 0.3 | | | | |
| 4 | 0 | 2.9 ± | 3.3 ± 0.9 | 3.0 ± 0.0 | 2.0 ± 0.0 | 0.0* |
| | | 1.3 | | | | |
| 5 | 0 | 2.0* ± | 2.2* ± | 3.0 ± 0.0 | 1.5 ± 0.5 | 0.0* |
| | | 0.0 | 0.4 | | | |
| 6 | 0 | 2.0* ± | 3.0 ± 0.0 | ND | 0.0* | ND |
| | | 0.0 | | | | |
| 7 | 0 | 2.9 ± | 3.5 ± 0.5 | 3.0 ± 0.0 | 1.9 ± 0.4 | 0.1* ± |
| | | 0.6 | | | | 0.2 |
| 8 | 0 | 3.3 | 3.9 | ND | ND | ND |

*Statistically significant (P < 0.05) from group 1.
ND = not determined.
Group 1 = Vehicle;
Group 2 = 0.5% NaMPA;
Group 3 = 1% NaMPA;
Group 4 = 2% NaMPA;
Group 5 = dexamethasone;
Group 6 = cyclosporine (IM);
Group 7 = no treatment;
Group 8 = Restasis.
Hyperemia scores represent the pooled average scores (+/−SEM) from both eyes of each animal in each group.

TABLE 15
Opacity Scores (pooled)

| | Days (score) | | | | | |
|---|---|---|---|---|---|---|
| Group | −2/−1 | 14/15 | 17/18 | 19/20 | 26/27 | 28/29 |
| 1 | 0 | 1.8 ± 1.7 | 2.8 ± 1.2 | 2.7 ± 0.7 | 1.7 ± 1.0 | 1.3 ± |
| | | | | | | 0.3 |
| 2 | 0 | 1.1 ± 1.0 | 3.5 ± 0.6 | 3.5 ± 0.7 | 2.1 ± 0.7 | 1.3 ± |
| | | | | | | 0.7 |
| 3 | 0 | 1.0 ± 0.9 | 3.1 ± 0.8 | 2.3 ± 0.3 | 2.1 ± 0.2 | 0.8 ± |
| | | | | | | 1.0 |
| 4 | 0 | 1.3 ± 1.2 | 2.5 ± 1.3 | 2.8 ± 0.9 | 2.4 ± 0.3 | 0.0* |
| 5 | 0 | 0.1* ± 0.3 | 1.1* ± | 2.0 ± 0.0 | 1.3 ± 0.5 | 0.0* |
| | | | 0.2 | | | |
| 6 | 0 | 1.3 ± 0.4 | 2.0 ± 0.0 | ND | 0.0* | ND |
| 7 | 0 | 1.7 ± 1.1 | 2.8 ± 0.8 | 2.6 ± 0.7 | 2.3 ± 0.4 | 0.4 ± |
| | | | | | | 0.6 |
| 8 | 0 | 2.2 | 3.3 | ND | ND | ND |

*Statistically significant (P < 0.05) from group 1.
ND = not determined.
Group designations are the same as for Table 13.

Opacity scores represent the pooled average scores (+/−SEM) from both eyes of each animal in each group.

### Example 8

### Studies Using Ovalbumin Induced Uveitis in Guinea Pigs

This study will assess the activity of the ocular solutions in a model of uveitis in guinea pigs. Uveitis is induced in animals by injecting ovalbumin into the footpad on Day 0. Treatment is given as described below. Ocular examination and histopathology is conducted to assess test article effects on inflammation, as described in more detail below.

The animals, 6 male guinea pigs/group, will be about 5-7 weeks of age at start of study, and housed either singly or in pairs, in HEPA-filtered shoe box cages, with bedding, feed and water given ad libitum, and exposed to a 12 hour light cycle.

The study will involve the following groups of animals: Group 1—Vehicle control; Group 2—Low dose NaMPA; Group 3—Mid dose NaMPA; Group 4—High dose NaMPA; Group 5—Dexamethasone positive control; Group 6—untreated control. In one type of treatment regimen, the groups of animals can be treated, as appropriate, with the ocular solutions beginning around Day 0 then continuing daily until the end of the experiment, around Day 30. The dosing regimen for the positive control group (group 5) may be different from that of the Vehicle control (group 1) and NaMPA groups (groups 2, 3, 4), including a shorter duration of dosing, beginning around Day 7 to Day 15 and continuing to around Day 30. In a second type of treatment regimen, animals can be treated, as appropriate, with the ocular solutions beginning around Day 7 to Day 15 then continuing daily until the end of the experiment, around Day 30. The dosing regimen for the positive control may be different from that of the Vehicle control and NaMPA groups, including shorter duration of dosing, beginning around Day 7 to Day 21 then continuing to around Day 30. In all types of treatment regimens, the frequency of dosing can be from once daily to eight times daily.

Uveitis is induced in the animals by injecting ovalbumin (Ova grade V, Sigma), conjugated with Imject alum (Thermal), subcutaneously into the foot pad on Day 0 and Day 7 (1 mg in a 100 μl volume). The animals are challenged with ovalbumin by administering ovalbumin eye drops (50 μg in PBS) on Day 14 (optionally may extend to day 21). Clinical observations are made at least once daily, and body weights are assessed prior to study, twice weekly, and at necropsy. Examination of anterior segment and fundoscopy will be performed daily beginning on Day 7 until necropsy.

Necropsy will be performed on Day 30, or as appropriate for the study. Necropsy involves removal of both eyes with either fixation in Modified Davidson's Solution, or fixation of one eye and freezing in OCT of the other eye. Blood will be collected following necropsy (1 ml of whole blood in EDTA, plasma, and frozen) for analysis.

Detailed and complete histopathologic assessment of all parts of the eye will be done, including scoring the severity and incidence of various inflammatory cell infiltrations. Special attention will be paid to cellular infiltration of conjunctiva and anterior segment. Cell infiltrations will be assessed using standard hematoxylin and eosin staining, or other known staining techniques (e.g., May Grunwald, Giemsa, PAS). Counting of various inflammatory cells will be performed blind by a pathologist, using a square reticle eye piece at a 400× magnification, and the cell count data analyzed for means and standard deviations for each of the study groups.

### Example 9

### Studies Based on Ragweed Induced Allergic Conjunctivitis

In this study, a murine model of active anaphylaxis was used to evaluate the efficacy of ocular NaMPA solutions on allergic conjunctivitis (Magnone et al., 1998, “A novel murine model of allergic conjunctivitis,” Clinical Immunology and Immunopathology. 87:75-84; Miyazaki et al., 2000, “Prevention of acute allergic conjunctivitis and late-phase inflammation with immunostimulatory DNA sequences,” Investigative Opthalmology and Visual Science 41:3850-3855). A schedule of experimental procedures is shown in Table 16.

TABLE 16
Schedule of Procedures

| | | | | Day | Day | Day | Day | Day | Day | Day | Day |
|---|---|---|---|---|---|---|---|---|---|---|---|
| Procedure | Day 0 | Day 1 | Day 2 | 21 | 22 | 23 | 24 | 25 | 26 | 27 | 28 |
| Health Checks | X | X | X | X | X | X | X | X | X | X | X |
| Weight Check | X | | | | | | | | | | X |
| Ocular Exam | X | | | X | | | | | | X | |
| Anesthesia | X | | | | | | | | | | |
| Photographs | | | | | | | | | | X | |
| Footpad | X | | | | | | | | | | |
| Injection | | | | | | | | | | | |
| Analgesic | X | X | X | | | | | | | | |
| Administration | | | | | | | | | | | |
| Dosing | | | | X | X | X | X | X | X | X | |
| Challenge | | | | | | | | | | X | |
| Behavior | | | | | | | | | | X | |
| Observations | | | | | | | | | | | |
| Euthanasia | | | | | | | | | | | X |
| Eye | | | | | | | | | | | X |
| Enucleations | | | | | | | | | | | |

Sensitization. Under ketamine (80 mg/kg)/xylazine (10 mg/kg) anesthesia, animals received footpad injections in both hind feet containing a suspension of 50 μg of short ragweed allergen (SRW, Greer, Lenoir, N.C., USA) and 1 mg Aluminum Hydroxide in 25 μL PBS.

Challenge. On day 27, after receiving the last dose of treatment, animals were challenged with topical doses of 1000 μg SRW suspension in 5 μl PBS in each eye. SRW was prepared fresh daily, mixed well before administration to ensure homogeneity, and used within 3 hours of mixing.

TABLE 17
Administration of Test and Control Articles

| | Number of | | | Dose | Volume |
|---|---|---|---|---|---|
| Group | Animals | | | Volume | per |
| Number | Male | Female | Test Article | per Day/eye | Dose |
| 1 | 0 | 8 | 2% NaMPA | 20 μL | 5 μL |
| 2 | 0 | 8 | 1% NaMPA | 20 μL | 5 μL |
| 3 | 0 | 8 | 0.5% NaMPA | 20 μL | 5 μL |
| 4 | 0 | 8 | 1% prednisolone | 20 μL | 5 μL |
| | | | acetate | | |
| | | | (Pred Forte ®) | | |
| 5 | 0 | 8 | Vehicle + sensitization | 20 μL | 5 μL |
| 6 | 0 | 8 | No treatment + | n/a | n/a |
| | | | sensitization | | |
| 7 | 0 | 8 | Vehicle + no | 20 μL | 5 μL |
| | | | sensitization | | |

NaMPA formulations. The ophthalmic NaMPA formulations used in this study consisted of 2%, 1% and 0.5% NaMPA (w/v) solutions prepared according to Table 1. NaMPA solutions were stored at 5° C.±3° C. in the dark.

Positive Control. The positive control used in this study consisted of 1% prednisolone acetate (Pred Forte®, Allergan, Lot #57284). Pred Forte® was stored at 25° C.±3° C. as per manufacturer's instructions.

Negative control. The negative control used in this study was the same formulation used to prepare the NaMPA solutions but lacking NaMPA. This negative control is referred to as “Vehicle”. Vehicle was stored at 5° C.±3° C.

Animals. Fifty-six (56) female Balb/C mice were used in this study (Harlan Laboratories). Mice were approximately 6-8 weeks of age upon arrival.

Dosing. On days 21-27, mice were dosed 4 times daily with NaMPA, positive control, or Vehicle in both eyes as specified in Table 17. Mice were dosed topically to the central cornea using a calibrated micropipette, with a 5 μL drop of treatment in each eye. On Day 27, mice received four doses and were challenged with SRW 15 minutes after the last dose.

Clinical Observations. Animals were observed daily, with special attention given to ocular findings such as redness, swelling, and discharge.

Animals were also observed 3, 5, 7, and 10 minutes after challenge on day 27 for rate of face-washing, indicating itching. At each of these time points the mice were observed for one full minute and graded according to the scale described in Grading Systems for Allergic Response.

Opthalmology. Ophthalmic exams were performed at baseline to verify that the eyes did not exhibit any signs of ocular irritation. Ophthalmic exams were repeated on the first day of dosing prior to the administration of the first dose and again on day 27, after the last dose. Exams were also performed on day 27, 15 minutes after the allergen challenge.

Tissue Collections and Preservation. Immediately after euthanasia using a lethal dose of sodium pentobarbital, all eyes were collected by excising out a section of each eye, including some surrounding tissue.

Histology and histopathology. Histological evaluation included density grading of the following cell types: eosinophils, neutrophils, CD4+ cells (i.e., CD4+ T cells) and macrophages. The grader was masked to the treatment group. Frozen tissue blocks were cut into 10 μm sections using a cryostat and stained with primary antibodies specific to each cell type as follows: anti-mouse major basic protein (eosinophils); anti-mouse NIMP-R14 (neutrophils); anti-mouse F4/80 (monocyte/macrophage lineage); anti-mouse CD4 (CD4+ cells). Following incubation with primary antibodies, sections were washed and then incubated with an appropriate fluorescently-labeled secondary antibody. Following a further washing step, sections were ready for viewing by immunofluorescent microscopy. Immunofluorescence was examined using an Olympus microscope at 200× magnification. Cells were manually counted in one eye of each animal in three separate fields in the fomiceal conjunctiva to a stromal depth of 300 μm parallel to the basement membrane of the surface epithelium. The average of the three fields was calculated for each eye.

Statistical analysis. Both eyes of each animal were averaged and all animals within a group were averaged to obtain an average score for each treatment group for each measurement parameter. Statistically significant differences between groups were determined using the 2-tailed, 2-sample t-test.

Results: Pre-challenge exams on Day 27 revealed that all 3 concentrations of NaMPA were well tolerated by the mice.

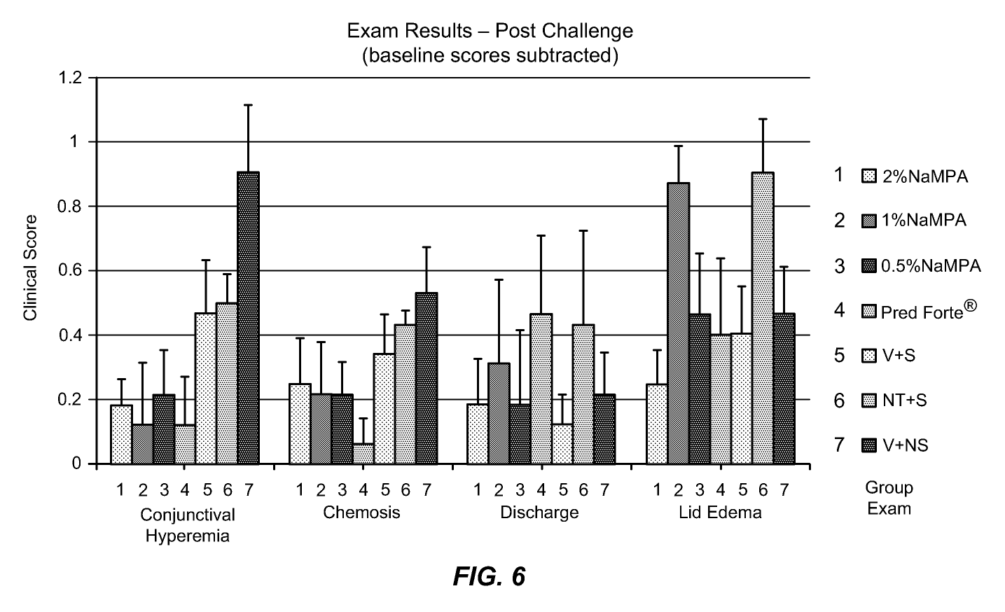

FIG. 6 shows post challenge results for conjunctival hyperemia, chemosis, discharge and eyelid edema. Data are shown as the average clinical scores (with standard error of the mean, SEM) for both eyes of all animals per treatment group, corrected for pre-challenge baseline score in each eye: V+S=Vehicle treated, SRW sensitized; NT+S=not treated, SRW sensitized; V+NS=Vehicle treated, not sensitized.

Clinical scores were graded on a 0-4 scale (see Grading Systems for Allergic Response), immediately before, and 15 minutes after being challenged with SRW.

As shown in FIG. 6, post-challenge exams revealed that there was less conjunctival hyperemia in all NaMPA and Pred Forte® treated groups (groups 1-4) than all other Vehicle or non-treated control groups (groups 5-7). This reduction was statistically significant for the 2.0% NaMPA group vs. groups 6 and 7 (p<0.05), and for the 1.0% and 0.5% NaMPA and Pred Forte® groups vs. group 7 (p<0.02). There was slightly less chemosis in the NaMPA treated groups than the Vehicle and non-treated control groups, but these decreases were not statistically significant. The Pred Fortei group presented with the lowest degree of chemosis, which was statistically significant vs. group 6 (p<0.001) and group 7 (p<0.02). There were no consistent differences in tearing/discharge between groups.

The 2% NaMPA group showed the lowest degree of lid edema than all other groups, including Pred Forte®. This reduction was statistically significant (p<0.01) vs. the non-treated/sensitized group (group 6).

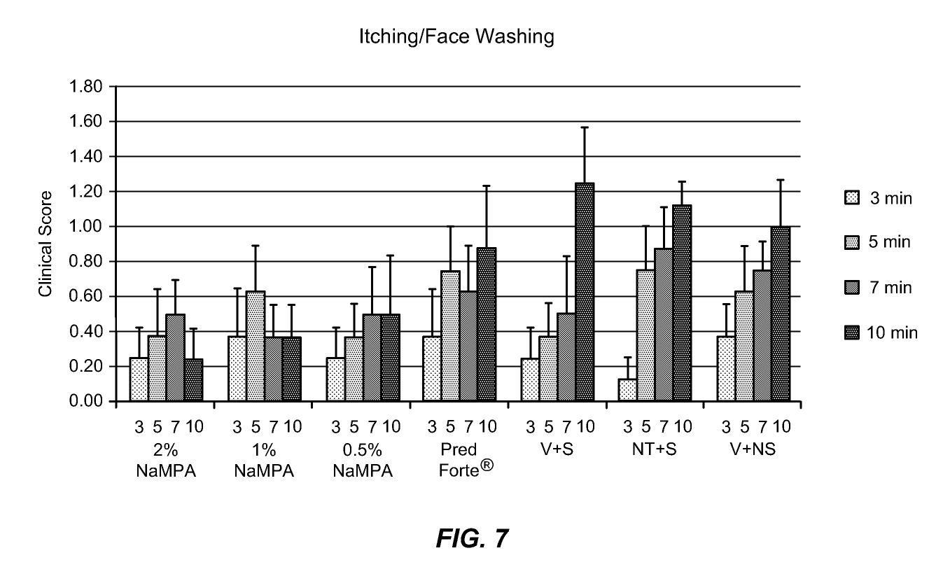

FIG. 7 shows itching and face washing test results. Data are shown as the average itching/face washing scores (with standard error of the mean, SEM) observed 3, 5, 7 and 10 minutes following challenge with SRW in both eyes. There was a statistically significant reduction in scoring at 10 minutes in the 2% NaMPA group vs. Vehicle (V+S p<0.02), vs. no treatment (T+S p<0.001) and vs. unsensitized (V+NS p<0.05) groups, and in the 1% NaMPA vs. Vehicle (p<0.05), and vs. no treatment (p<0.01) groups: As shown in FIG. 7, treatment with all three concentrations of NaMPA resulted in a lower incidence of itching/face-washing than all other groups, particularly at the 10 minute time point, when itching/face-washing increased dramatically in all the non-NaMPA groups, including Pred Forte®. Itching/face washing in the Pred Forte® group was slightly lower than the other control groups, but this difference was not statistically significant.

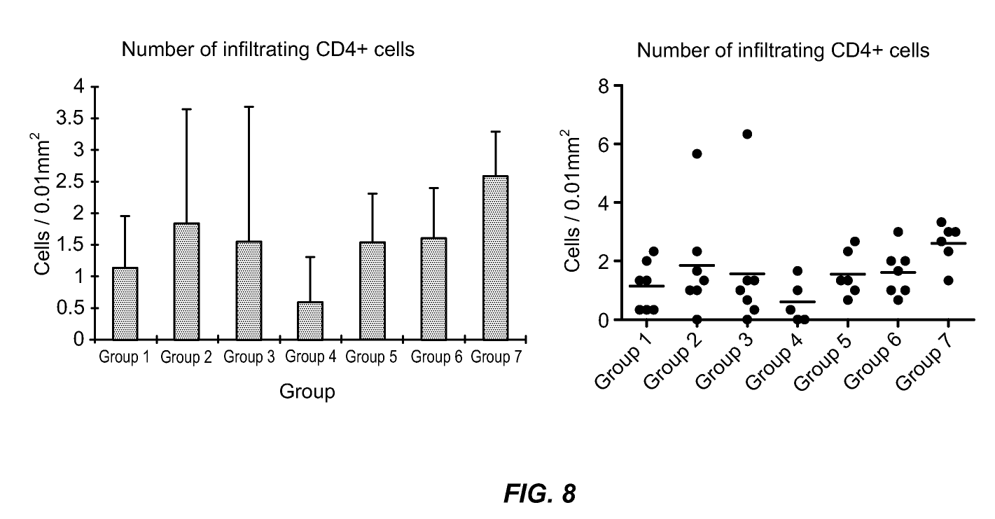

FIG. 8 shows the numbers of infiltrating CD4+ cells in the conjunctiva following SRW challenge. Data are average number of CD4+ cells in 3 separate fields. Group averages are shown in the left-hand figure; individual data (average of 3 fields) from each animal are shown on the right.

As shown in FIG. 8, the numbers of infiltrating CD4+ cells (i.e., CD4+ T cells) were lowest in the 2% NaMPA and in the Pred Forte® groups. These decreases were statistically significant for the 2% NaMPA group vs. group 7 (V+NS; p<0.01), and between the Pred Forte® group and groups 6 (T+S; p<0.05) and 7 (p<0.01).

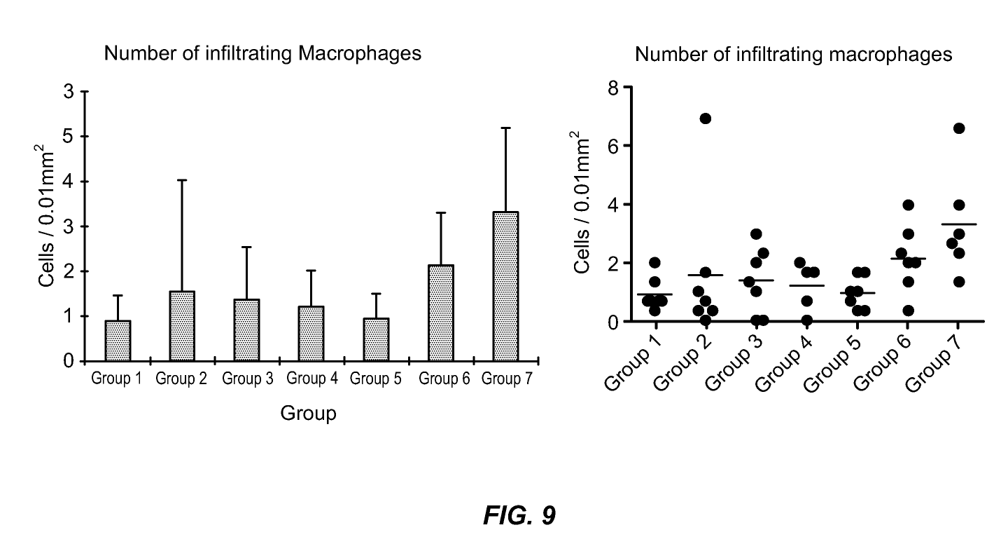

FIG. 9 shows the numbers of infiltrating macrophages in the conjunctiva following SRW challenge. Data are average number of macrophages in 3 separate fields. Group averages are shown in the left-hand figure; individual data (average of 3 fields) from each animal are shown on the right. There was a statistically significant reduction in numbers of infiltrating macrophages in group 1 (2% NaMPA) vs. group 6 (T+S; p<0.05) and group 7 (V+NS; p<0.01), and between group 4 (Pred Forte®) and group 7 (p<0.05).

NaMPA, even at the highest concentration used in this study, did not produce any notable ocular irritation or adverse clinical signs, and was well tolerated by the mice. Statistically significant decreases in itching/face washing behavior, in multiple parameters of the clinical response, and in numbers of infiltrating inflammatory cells were observed in NaMPA treated groups in response to topical SRW challenge. This model and its modifications have been demonstrated in multiple laboratories to be effective at demonstrating allergic conjunctivitis and have served to help elucidate the immunological mechanisms underlying ocular allergy (Magone et al., 1998, Clin Immunol Immunopath 87:75-84; Groneberg et al., 2003, Allergy 58: 1101-1113; Miyazaki et al., 2000, Invest Opthalmol Vis Sci 41:3850-3855; Fukushima et al., 2006, Mol Vision 12:310-317; Fukushima et al., 2005, J Immunol 175: 4897-4903; Stern et al., 2005, Invest Opthalmol Vis Sci 46: 3239-3246). The Balb/C strain of mice has been shown in multiple studies to be suitable in this model (Fukushima et al 2006; Fukushima et al. 2005; Stem et al. 2005). Collectively, this data indicates that ophthalmic NaMPA formulations are effective in reducing clinical signs and histopathological findings in a model of allergic conjunctivitis, and therefore have considerable potential for the prevention and treatment of allergic conjunctivitis in humans.

### Example 10

### Studies Using the Rabbit Model of Compound 48/80 Induced Signs of Allergic Conjunctivitis

This study evaluated the effectiveness of ocular NaMPA solutions in preventing the development of ocular signs of an allergic response, using a model of allergic conjunctivitis induced by Compound 48/80 in New Zealand White (ZW) rabbits.

Ocular allergy is mediated primarily by mast cells, which are immune cells that contain pro-inflammatory mediators. Upon degranulation by cross-linking of IgE, the mast cell releases histamine, prostaglandins, leukotrienes, chemotactic factors, interleukins, as well as other cytokines and vasoactive amines. Some of these substances, e.g., histamine and prostaglandins, directly affect blood vessels and nerves, whereas others result in the migration of inflammatory cells such as neutrophils, eosinophils and macrophages. Together, these mediators cause the signs and symptoms of ocular allergy.

Compound 48/80 is a condensation product of N-methyl-p-methoxyphenethylamine and formaldehyde, and initiates mast cell degranulation without antigen-antibody binding. It is widely used as a preliminary screen for potential anti-allergic compounds (Abelson et al., 1983, “Conjunctival Eosinophils in compound 48/80 rabbit model,” Arch Opthalmol. 101:631-633; Khosravi et al., 1995, “Allergic conjunctivitis and uveitis models: Reappraisal with some marketed drugs,” Inflamm Res. 44:47-54; Udell et al., 1981, “Animal and Human ocular surface response to a topical nonimmune mast-cell degranulating agent (compound 48/80),” Amer Jour Opthalmol. 91:226-230). In addition, Compound 48/80 has been shown to be particularly effective at inducing degranulation of conjunctival mast cells as opposed to mast cells located in the human nasal mucosa, skin and other tissues. (See, Abelson et al, supra; Church et al., 1991, “Biological properties of human skin mast cells,” Clin Exp Allergy 21:Suppl 3:1-9).

NaMPA. The ophthalmic NaMPA formulations used in this study consisted of 2%, 1% and 0.5% NaMPA (w/v) solutions prepared according to Table 1. NaMPA solutions were stored at 5° C.±3° C. in the dark.

Positive Control. The positive control used in this study consisted of 1% prednisolone acetate (Pred Forte®, Allergan, Lot #57284). Pred Forte® Q was stored at 25° C.±3° C. as per manufacturer's instructions.

Negative control. The negative control used in this study was the same formulation used to prepare the NaMPA solutions but lacking NaMPA. This negative control is referred to as “Vehicle”. Vehicle was stored at 5° C.±3° C.

Animals. Thirty six (36) New Zealand White (ZW) adult male rabbits (Oryctolagus cuniculus) were used in this study. Rabbits were approximately 1.5-2.5 kg by weight and at least 11 weeks old upon arrival. Rabbits were obtained from a registered commercial breeder.

TABLE 18
Schedule of Procedures

| | Day | Day | Day | Day | Day | Day | Day | Day |
|---|---|---|---|---|---|---|---|---|
| Procedure | 1 | 2 | 3 | 4 | 5 | 6 | 7 | 8 |
| Health Checks | X | X | X | X | X | X | X | X |
| Weight Checks | X | | | | | | | X |
| Ocular Exams | X | | | | | | X | |
| Photographs | X | | | | | | X | |
| Dosing | X | X | X | X | X | X | X | |
| Challenge | | | | | | | X | |
| Euthanasia | | | | | | | | X |
| Enucleations | | | | | | | | X |

Compound 48/80 challenge. On day 7, fifteen minutes after receiving the last dose of treatment, animals were challenged with topical doses of 25 μl of 30 mg/mL of Compound 48/80 in both eyes using a calibrated micropipette. Compound 48/80 was prepared fresh on day of challenge, used within 3 hours of mixing, and mixed well before administration to ensure homogeneity.

TABLE 19
Administration of Test and Control Articles

| | | | |
|---|---|---|---|
| | Number of | Dose | Volume |
| Group | Animals | Volume | per |

| No. | Males | Females | Test Article | per Day/Eye | Dose |
|---|---|---|---|---|---|
| 1 | 6 | 0 | 2.0% NaMPA | 160 μL | 40 μL |
| 2 | 6 | 0 | 1.0% NaMPA | 160 μL | 40 μL |
| 3 | 6 | 0 | 0.5% NaMPA | 160 μL | 40 μL |
| 4 | 6 | 0 | 1% prednisolone | 160 μL | 40 μL |
| | | | acetate | | |
| 5 | 6 | 0 | Vehicle | 160 μL | 40 μL |
| 6 | 6 | 0 | No treatment | n/a | n/a |

Dosing. On days 1-7, rabbits were dosed 4 times daily with NaMPA, positive control, or Vehicle topically in both eyes, as specified in Table 19. Rabbits were dosed using a calibrated micropipette, with a 40 μL drop of treatment in each eye. On day 7, animals received four doses and were challenged 15 minutes after the last dose. All doses were delivered with at least 2 hours between doses.

Opthalmology. Ophthalmic exams were performed at baseline (study entry) to verify that the eyes did not exhibit any signs of ocular irritation. They were again examined on day 1 after the 4th dose was administered to test for tolerability in the animals.

On day 7 exams were repeated immediately prior to, and 5, 10, 15, 20, and 30 minutes post challenge. Scoring was done according to Grading Systems of the Allergic Response. Clinical signs (conjunctival injection in three vessels beds, chemosis, and discharge) were graded.

Tissue Collections and Preservation. Eyes were enucleated immediately following sacrifice and fixed in Alcoholic Bouin's/Duboscq-Brazil Fluid fixative for at least 10 hours, but not more than 24 hours, and then transferred to 70% ethanol. The tissue was embedded and sectioned (5 μm thick) through the central vertical plane, and stained for evaluation.

Statistical Analysis. Scoring data for both eyes of each animal were averaged and data for all animals within a group were averaged to obtain an average score for each treatment group for each measurement parameter. Statistically significant differences between groups were determined using the 2-tailed, 2-sample t-test.

Results: NaMPA, even at the highest concentration used in this study, did not produce any notable ocular irritation or adverse clinical signs, when administered topically four times daily for seven days, and was well tolerated by the rabbits.

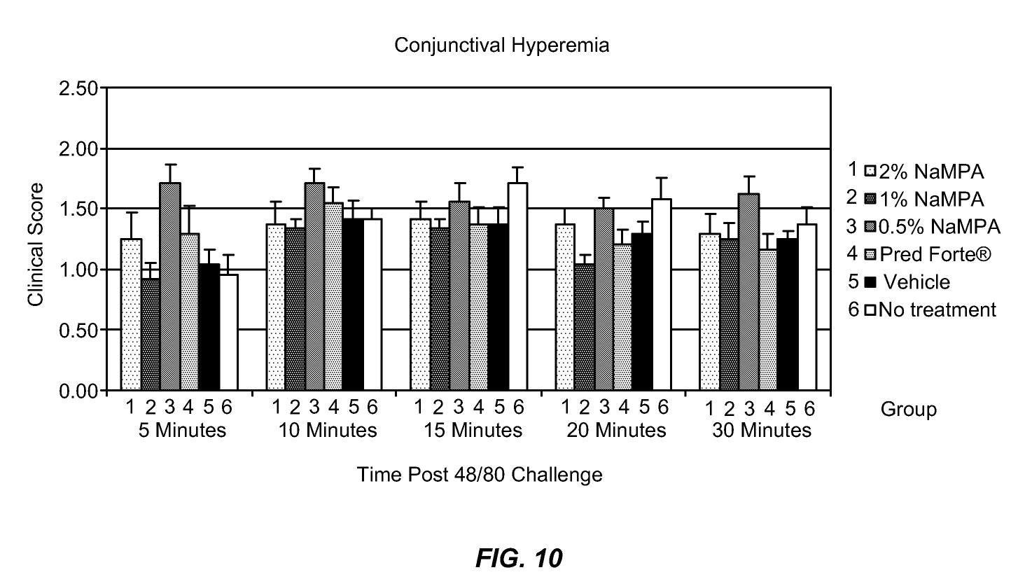

FIG. 10 shows analysis of conjunctival hyperemia. Data are mean conjunctival hyperemia scores (scale=0-4) 5, 10, 15, 20, and 30 minutes after topical challenge with Compound 48/80 in both eyes.

As shown in FIG. 10, slightly less conjunctival hyperemia was present in the 1.0% NaMPA group at most time points when compared to all other treatment groups (FIG. 2). The reduction was statistically significant vs. the no treatment group at 20 and 30 minutes post-challenge (p<0.05). At 10, 20, and 30 minutes postchallenge the Pred Forte® group had a statistically significant reduction in lower episcleral hyperemia vs. Vehicle.

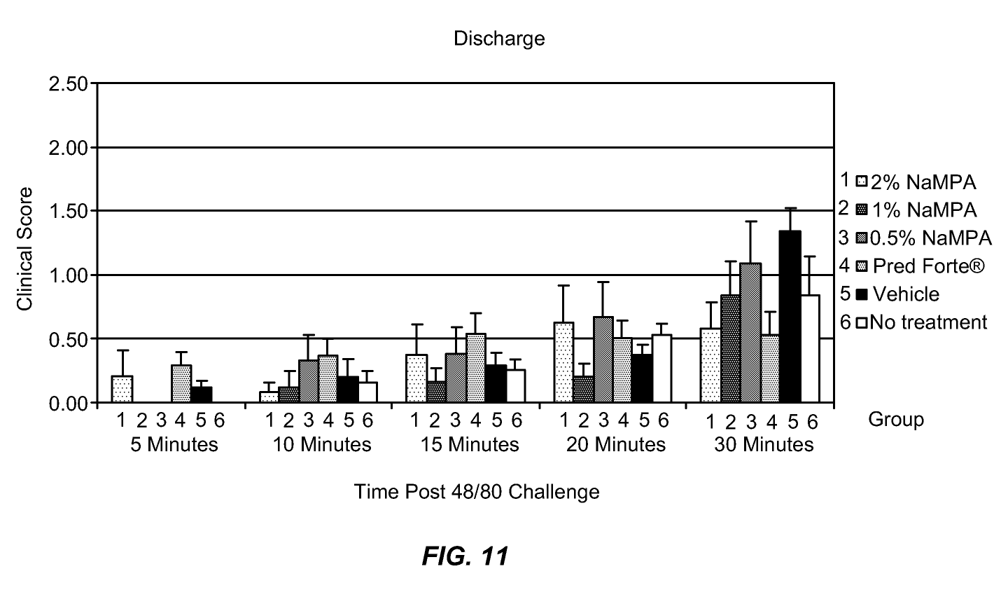

FIG. 11 shows discharge scores. Data are mean discharge scores (scale=0-4) 5, 10, 15, 20, and 30 minutes after topical challenge with Compound 48/80 in both eyes. There was a statistically significant decrease in the 1.0% NaMPA group vs. the untreated group at 20 min (P<0.05) and in the 2% NaMPA group vs. the Vehicle group at 30 min (p<0.05), and in the Pred Forte® group vs. the Vehicle group at 30 min (p<0.02).

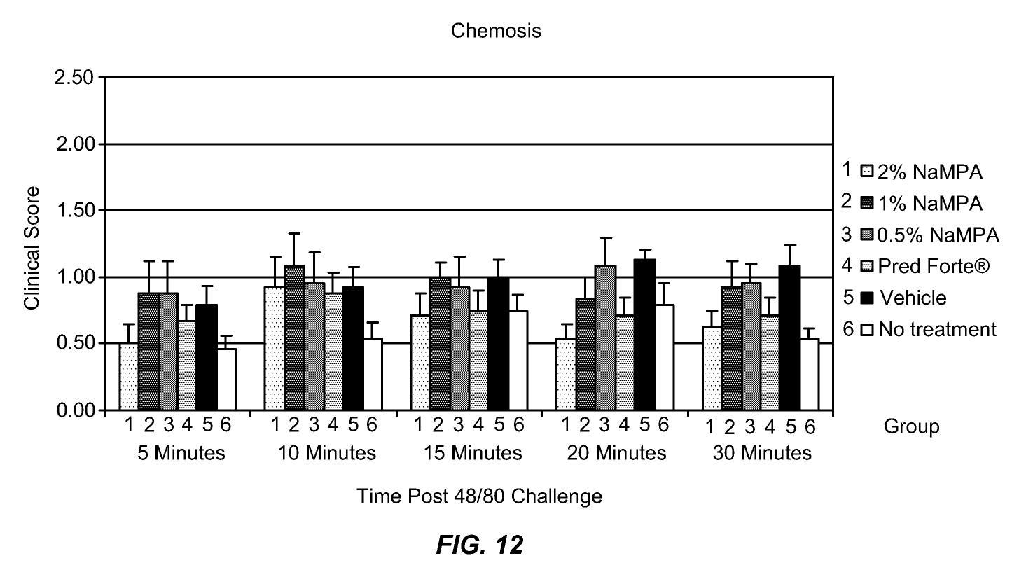

FIG. 12 shows chemosis analysis. Data are mean chemosis scores (scale=0-4) 5, 10, 15, 20, and 30 minutes after topical challenge with Compound 48/80 in both eyes. There was a statistically significant reduction in the 2% NaMPA group vs. the Vehicle group at 20 min (p<0.001), and at 30 min (p<0.05), and in the Pred Forte® group vs. Vehicle at 20 min (p<0.05).

NaMPA, even at the highest concentration used in this study, did not produce any notable ocular irritation or adverse clinical signs, when administered topically four times daily for seven days, and was well tolerated by the rabbits.

These results indicate that ophthalmic NaMPA formulations were effective in reducing the clinical signs of conjunctival hyperemia, chemosis and discharge induced by Compound 48/80 in the rabbit. This could be an indication of an anti-inflammatory effect of NaMPA. and also suggests that NaMPA may be effective in reducing inflammation associated with the late phase of the allergic response, where lymphocytes (such as CD4+ T cells) may play a prominent role. Decreases in these parameters with the two highest concentrations of NaMPA indicate that perhaps a more definitive anti-inflammatory effect might be seen with higher concentrations of NaMPA, extended dosing regimens and/or different formulations.

In summary, the results demonstrate the effectiveness of ophthalmic NaMPA formulations in multiple models of ocular inflammatory disease. Using standard statistical analysis of the data, efficacy was demonstrated by clinical observation (e.g. conjunctival hyperemia, lid edema, itching/face washing, chemosis, discharge), functional assessment (e.g., TBUT) or histological assessment (tissue pathology, cell infiltration). NaMPA was highly effective in models of dry eye and allergic conjunctivitis, in many instances equivalent to or exceeding the efficacy of the positive controls dexamethasone and Restasis®. In addition, NaMPA activity was demonstrated in a model of anterior uveitis based on histopathological findings. In every model and species tested, topical administration of ophthalmic NaMPA formulations was well tolerated under normal conditions and during clinical signs of ocular inflammatory disease. Taken together, the data indicates that topical administration of ophthalmic NaMPA formulations has beneficial effects on the clinical signs, ocular function and histopathology of multiple eye tissues in these models of ocular inflammatory disease. These findings are consistent with the data presented in FIGS. 1, 2, 3 and Table 4 demonstrating penetration of multiple ocular tissues by ophthalmic NaMPA formulations following topical administration.

### Example 11

### Grading Systems for Allergic Response

The following grading systems were used for assessing allergic responses in the foregoing examples.

Conjunctival Hyperemia (Graded in Conjunctival, Ciliary, Episcleral Vessel Beds)
0.0 Blood vessels normal, conjunctiva may appear without perilimbal injection
1.0—Slight localized injection
2.0—Deeper crimson injection of vessels covering less than half of the white area of conjunctiva
3.0—Injection covering majority of conjunctiva but there are still visible areas of white. Still able to differentiate somewhat between blood vessels
4.0—Diffuse beefy red injection covering almost entire conjunctiva. Almost impossible to discern different blood vessel

Chemosis
0.0—No swelling of conjunctiva
1.0—Slight diffuse or regional swelling of conjunctiva. Vessels still highly visible.
2.0—Definite general swelling of conjunctiva. Vessels still discernable
3.0—Very pronounced swelling of conjunctiva. Difficulty seeing deep vessels.
4.0—Opaque conjunctiva. Total separation between blood vessels. Superficial vessels unable to be seen. Bulging and swelling of nictitating membrane and tarsus.

Tearing/Discharge
1.0—slight projection of tear meniscus
2.0—moderate projection of tear meniscus or tearing
3.0—prominent projection of tear meniscus or tearing
4.0—prominent projection of tear meniscus and significant tearing out of the eye

Lid Edema
1.0—Lid margins slightly bulged
2.0—Slight decrease of palpebral fissure
3.0—Significant decrease of palpebral fissure, not closed
4.0—Lids swollen shut

Face-Washing/Itching
0—No itching/face-washing
2—>10 swipes at head per 5 seconds, >3 times per minute
1—>10 swipes at head per 5 seconds, <3 times per minute
   *Note-0.5 units are allowed for any ocular score.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. An ocular solution consisting essentially of:
sodium mycophenolic acid (NaMPA), wherein the pH of the solution is from about 6.0 to about 8.5.

2. An ocular solution consisting essentially of:
sodium mycophenolic acid (NaMPA), wherein the pH of the solution is from about 6.0 to about 8.5; and one or more additives selected from a preservative, viscosity enhancing agent, wetting agent, antioxidant, buffering agent lubricating agent, and tonicity agent.

3. The ocular solution of claim 1 or 2 in which the sodium mycophenolic acid is from about 0.01% w/v to about 4.0% w/v.

4. The ocular solution of claim 1 or 2 in which the sodium mycophenolic acid is from about 0.05% w/v to about 3.0% w/v.

5. The ocular solution of claim 1 or 2 in which the sodium mycophenolic acid is from about 0.1% w/v to about 2.0% w/v.

6. The ocular solution of claim 1 or 2 in which the sodium mycophenolic acid is from about 1.0% w/v to about 4.0% w/v.

7. The ocular solution of claim 1 or 2 in which the pH is from about 7.0 to about 8.0.

8. The ocular solution of claim 1 or 2 in which the pH is from about 7.0 to about 7.5.

9. The ocular solution of claim 1 or 2 in which the total sodium in the solution is about 0.4 to about 2.0% w/v.

10. The ocular solution of claim 1 or 2 in which the total sodium in the solution is about 0.9% w/v.

11. The ocular solution of claim 2 in which the additive is a preservative selected from benzalkonium chloride, benzethonium chloride, benzododecinium bromide, cetylpyridinium chloride, chlorobutanol, ethylenediamine tetracetic acid (EDTA), thimerosol, phenymercuric nitrate, phenylmercuric aceteate, methyl/propylparabens, phenylethyl alcohol, sodium benzoate, sodium propionate, sorbic acid, and sodium perborate.

12. The ocular solution of claim 2 in which the additive is a viscosity enhancing agent selected from carbopol gels, carboxymethycellulose, dextran, gelatine, glycerin, hydroxyethyl cellulose, hydroxypropyl methylcellulose, methylcellulose, ethylcellulose, polyethylene glycol, poloxamer 407, polysorbate 80, propylene glycol, polyvinyl alcohol, and polyvinylpyrrolodine (povidone).

13. The ocular solution of claim 2 in which the additive is a buffering agent selected from acetate, borate, phosphate, bicarbonate, carbonate, citrate, tetraborate, biphosphate, tromethamine, hydroxyethyl morpholine, and trishydroxymethylamino-methane (THAM).

14. The ocular solution of claim 2 in which the additive is a tonicity agent selected from dextran 40, dextran 70, dextrose, glycerin, potassium chloride, propylene glycol, and sodium chloride.

15. The ocular solution of claim 2 in which the additive is a wetting agent selected from polysorbate 20 and 80, poloxamer 282, tyloxapol, hydroxypropylmethyl cellulose, carboxymethylpropyl cellulose, povidone, and polyvinyl alcohol.

16. The ocular solution of claim 2 in which the additive is a lubricating agent selected from propylene glycol, ethylene glycol, polyethylene glycol, hydroxypropylmethylcellulose, carboxymethylcellulose, hydroxypropylcellulose, dextran 40, dextran 70, gelatin, polyvinyl alcohol, polyvinylpyrrolidone, povidone, petrolatum, mineral oil, and a carbomer.

17. The ocular solution of claim 2 in which the additive is a phospholipid-based lubricating agent.

18. The ocular solution of claim 1 or 2 in which sodium counter ion is chloride.

19. The ocular solution of claim 18 in which the chloride is from HCl.

20. A method of treating an ocular disorder associated with an inflammatory or autoimmune condition, the method comprising administering topically the solution of claim 2 to an affected eye.

21. The method of claim 20 in which the ocular disorder affects the front of the eye.

22. The method of claim 21 in which the ocular disorder affecting the front of the eye is selected from blepharitis, keratitis; rubeosis iritis; Fuchs' heterochromic iridocyclitis; chronic uveitis or anterior uveitis; conjunctivitis; allergic conjunctivitis; keratoconjunctivitis sicca; iridocyclitis; iritis; scleritis; episcleritis; corneal edema; scleral disease; ocular cicatrcial pemphigoid; pars planitis; Posner Schlossman syndrome; Behçet's disease; Vogt-Koyanagi-Harada syndrome; conjunctival edema; conjunctival venous congestion; periorbital cellulitis; acute dacryocystitis; non-specific vasculitis; and sarcoidosis.

23. The method of claim 20 in which the ocular disorder affects the back of the eye.

24. The method of claim 23 in which the ocular disorder affecting the back of the eye is selected from macular edema, cystoid macular edema; retinal ischemia; and choroidal neovascularization; macular degeneration; diabetic retinopathy; diabetic retinal edema; retinal detachment; uveitis; panuveitis; choroiditis; episcleritis; scleritis; Birdshot retinochoroidopathy; retinal vasculitis; choroidal vascular insufficiency; choroidal thrombosis; optic nerve neovascularization; and optic neuritis.

25. The method of claim 20 in which the ocular disorder is uveitis.

26. The method of claim 20 in which the ocular disorder is allergic conjunctivitis.

27. The method of claim 20 in which the ocular disorder is keratoconjunctivitis sicca.

28. The method of claim 20 in which the solution is administered one to four times daily.

29. The method of claim 20 in which the solution is administered once every two days.

30. The method of claim 20 in which the solution is administered once every four days.

31. The method of claim 20 in which the solution is administered once every week.
